(19) United States
(12) Patent Application Publication
(10) Pub. No.: US 2004/0258676 A1
(43) Pub. Date: Dec. 23, 2004

(54) Stimulation of the activity of an isoform of lysyl oxidase for combating against some pathologies due to an incomplete, absent or disorganized elastogenesis

(75) Inventors: Eric Perrier, Les Cotes d'Arey (FR); Valerie Cenizo, Vaulx-Milieu (FR); Charbel Bouez, Lyon (FR); Pascal Sommer, Saint Genis Laval (FR); Odile Damour, Saint Genis Laval (FR); Claudine Gleyzal, Lyon (FR); Valerie Andre, Ampuis (FR); Corinne Reymermier, Charly (FR); Isabelle Orly, Irigny (FR)

Correspondence Address: MERCHANT & GOULD PC, P.O. BOX 2903, MINNEAPOLIS, MN, 55402-0903, US

(73) Assignees: COLETICA, Lyon (FR); CENTRE NATIONAL DE LA RECHERCHE, Paris (FR)

(21) Appl. No.: 10/852,575

(22) Filed: May 24, 2004

(30) Foreign Application Priority Data
Jun. 13, 2003 (FR) 03 07178

(51) Int. Cl. A61K 38/44

(52) U.S. Cl. 424/94.4

(57) ABSTRACT

The invention relates to the stimulation of the activity of an isoform of lysyl oxidase, and more particularly of the LOX (lysyl oxidase) isoform.

The invention relates notably to a screening method of an active principle which regulates elastogenesis in cases of pathological, disorganized and/or non-functional elastogenesis, as in cases of fibrosis, of solar elastosis, of stretch marks, and/or of dystrophic scars; and/or in cases of some eczematous pathologies.

The aim of the invention is mainly to provide such a screening method so as to provide compositions enabling the elastogenesis in the cases cited to be regulated.

The invention relates to the stimulation of the activity of an isoform of lysyl oxidase, and more particularly of the LOX (lysyl oxidase) isoform.

### STATE OF THE ART

The properties of resistance and of elasticity of the skin and of the mucous membranes are essentially defined by the collagen fibers and elastin fibers of the dermis. Elastin is a protein which makes up the elastic fibers which are also constituted by other molecules such as fibrillins and MAGPs (Microfibrillar Associated Glycoproteins).

The elastic fibers are formed of elastin deposited on the microfibrils. Elastin is synthesized in the form of—soluble tropoelastin which acquires its physico-chemical properties (insolubility, elasticity) after the intra- and inter-molecular cross-linking of it by a lysyl oxidase, and then its deposit on the microfibrils. The elastic fibers are responsible for the elastic property of the organs which contain them (vessels, pulmonary parenchyma, elastic cartilages, skin . . . ). The elastic fibers are mainly constituted of elastin deposited on the microfibrils. The name <<elastin >> was reserved to the protein which forms the amorphous portion of the elastic fibers and which imparted their elasticity to them.

Recently, components of these elastic fibers have been shown in the epidermis.

The collagen fibrils are formed by the trimeric assembly of chains of collagen. These collagen fibers are also cross-linked by the lysyl oxidase LOX. The collagen fibers are synthesized in the connective tissues throughout the whole life. Five known isoforms exist in the family of lysyl oxidases (LOs): LOX, LOXL, LOXL2, LOXL3, LOXL4 (Csiszar, Lysyl oxidases: A novel multifunctional amine oxidase family, Nucleic Acid Research and Molecular Biology, 2001, vol 70, p2-28). LOX is involved in the cross-linking of the collagen fibers (Seve et al., Expression analysis of recombinant lysyl oxidase (LOX) in myofibroblast-like cells, Connective Tissue Research, 2002, 43: 613-619). LOX is also associated with the upkeep of cellular homeostasis and may be considered as a regulator which is necessary for the upkeep of cellular homeostasis through the regulation of NFkB (Jeay S, Pianetti S, Kagan H M, Sonenshein G E. Lysyl oxidase inhibits ras-mediated transformation by preventing activation of NF-kappaB. Mol Cell Biol 23:2251-63, 2003).

The synthesis of collagen is concomitantly deregulated in cases of pathological, disorganized and/or non-functional elastogenesis, such as fibrosis or solar elastosis, and in cases of some cancerous pathologies. The synthesis of collagen without expression of LOX is associated with pathological modifications of the extracellular matrix as in the case of cancers, unlike the synthesis of collagen and of LOX is associated with a densification of the matrix as a mechanism of defence against cell invasion (Decitre et al., Lab. Invest., 78, 143-151, 1998).

The synthesis of elastin is deregulated in cases of pathological, disorganized and/or non-functional elastogenesis, such as fibrosis or solar elastosis, and in cases of some cancerous pathologies. This anarchical synthesis of elastin leads to the formation of derived peptides which are in general considered as being favourable to the tumoral potential of the cells (Brassart B, Fuchs P, Huet E, Alix A J, Wallach J, Tamburro A M, Delacoux F, Haye B, Emonard H, Hornebeck W, Debelle L. Conformational dependence of collagenase (matrix metalloproteinase-1) up-regulation by elastin peptides in cultured fibroblasts. J. Biol. Chem. 276(7): 5222-7, 2001).

The prior art does not enable providing criteria which enable evaluating the impact of active principles on dermato-cosmetology enabling the regulation of elastogenesis. In this context, it is also very difficult to obtain objective criteria enabling the impact of these active to be judged. The methods of identification of actual active principles bear upon the evaluation of the expression of the genes involved in the formation of the elastic fibers, such as elastin or the fibrillins.

Furthermore, at the present time, animal experimentation is forbidden in cosmetics in Europe and human experimentation is ethically disputed. It is therefore unacceptable to the inventors to carry out a screening method, for cosmetic applications, which makes use of animals or human beings.

In three-dimensional cell models, such as Mimeskin® (Coletica, France), keratinocytes induce the synthesis of tropoelastin and the deposit of tropoelastin on the microfibrils (Duplan-Perrat et al., Keratinocytes influence the maturation and organization of the elastin network in a skin equivalent. J. Invest. Dermatol. 114: 365-70, 1999). In the Mimeskin® model, the extracellular matrix showed an ultra-structural organization similar to that of the skin, with the collagen being organized in rays and elastic fibers which are constituted of elastin deposited on the microfibrils. This model has also been used for testing the effectiveness of certain-molecules, such as inhibitors of-lysyl oxidases. This has enabled showing that the inhibition of the lysyl oxidases induced a disorganization of the collagen fibers and the elastin fibers, but also a deviation from the program of differentiation of the keratinocytes, with a reduction of the level of expression of the labels of terminal differentiation, such as filaggrin (French patent 01 10443, CNRS, Use of inhibitors of lysyl oxidases for cell culture and tissue engineering (<<Utilisation d'inhibiteurs des lysyl oxydases pour la culture cellulaire and le genie tissulaire >>). In that patent, no distinction is made between the different isoforms of LO.

Those studies did not enable developing a screening method of active principles which stimulate the expression of LOX and which down-regulate elastogenesis in cases of pathological, disorganized and/or non-functional elastogenesis, as in cases of fibrosis, of solar elastosis, of stretch marks, and/or of dystrophic scars and cancerous pathologies.

The prior art does not therefore enable providing active principles which stimulate the expression of LOX and which down-regulate elastogenesis in cases of pathological, disorganized and/or non-functional elastogenesis, as in cases of fibrosis, of solar elastosis, of stretch marks, and/or of dystrophic scars.

Furthermore, the prior art does not enable precisely locating the zone of expression of the isoform of the lysyl oxidase LOX, notably due to the fact that the methods provided by the prior art are imprecise and are not sufficiently specific about the various isoforms of lysyl oxidase.

### AIMS OF THE INVENTION

The aim of the invention is mainly to solve the technical problems set forth above and notably the technical problem aiming to provide a screening method of active principles which stimulate the expression of LOX or of an homologous or essentially homologous form thereof for regulating elastogenesis in cases of pathological, disorganized and/or non-functional elastogenesis, as in cases of fibrosis, of solar elastosis, of stretch marks, and/or of dystrophic scars. By “functional elastic fibers” is meant the usual meaning in the art as described above and notably in the context of the invention, elastic fibers which have elastic properties coming from a tridimensional structure.

The invention also relates to the use of the isoform of the lysyl oxidase LOX and of an active principle which inhibits the expression of the protein elastin, notably for regulating elastogenesis in cases of pathological, disorganized and/or non-functional elastogenesis, as in cases of fibrosis, of solar elastosis, of stretch marks, and/or of dystrophic scars. The effect obtained is thus synergistic.

The invention also relates to the use of an active principle which stimulates the enzymatic activity or the expression of the isoform of the lysyl oxidase LOX, said active principle inhibiting the expression of the protein elastin, or being combined with a second active principle which inhibits the expression of the protein elastin, notably for regulating elastogenesis in cases of pathological, disorganized and/or non-functional elastogenesis, as in cases of fibrosis, of solar elastosis, of stretch marks, and/or of dystrophic scars. The effect obtained is thus synergistic.

The invention also enables solving the technical problem consisting of providing an effective method of locating the expression of LOX and of tracking this expression.

### SUMMARY OF THE INVENTION

In this text, by the term “LOX”, or “hLOX”, the inventors mean the isoform of the human lysyl oxidase protein LOX having the sequence ID No 1.

In this text, by the term “elastin”, the inventors mean the human tropoelastin protein or the elastin protein having the sequence ID No 3.

In this text, by the term “collagen”, the inventors mean the various types of collagen which are presentin the human cutaneous tissue.

In this text, by the term “LOXL”, or “hLOXL”, the inventors mean the isoform-like (L) of the human lysyl oxidase protein LOXL having the sequence ID No 6.

By “stimulating the expression of the isoform of lysyl oxidase LOX”, the inventors mean the stimulation of the expression of the gene encoding LOX, and notably the stimulation of the synthesis of messenger RNA encoding LOX, but also the stimulation of the protein LOX which is synthesized from this messenger RNA. The LOX cDNA having the sequence ID NO ₂.

By “inhibiting the expression of the protein elastin”, the inventors mean the inhibition of the expression of the gene encoding the protein elastin, and notably the inhibition of the synthesis of messenger RNA encoding the protein elastin, but also the inhibition of the synthesis of the protein elastin or of its precursor tropoelastin, from this messenger RNA. The cDNA of the protein tropoelastin having the sequence ID No 4.

By “stimulating the expression of collagen I”, the inventors mean the stimulation of the expression of the gene encoding collagen I alpha1, and notably the stimulation of the synthesis of Messenger RNA encoding collagen 1 alpha1, but also the stimulation of the synthesis of collagen I, or of its precursor procollagen I, from this messenger RNA.

This stimulation of the expression of LOX must be carried out in a manner sufficiently effective in order to contribute to regulating elastogenesis in cases of pathological, disorganized and/or non-functional elastogenesis, as in cases of fibrosis, of solar elastosis, of stretch marks, and/or of dystrophic scars. In some pathologies, it is desirable to inhibit the expression of the protein elastin so as to obtain an effective effect which regulates disorganized elastogenesis which is observed notably in the case of solar elastosis or of fibrosis.

In this invention, the inventors thus aim to stimulate either the expression of LOX or of an homologous or essentially homologous form thereof, or the enzymatic activity of LOX or of an homologous or essentially homologous form thereof, and to inhibit the expression of the protein elastin.

By “an homologous or essentially homologous form thereof” it is meant an homologous form of the isoform of lysyl oxidase LOXL which has the same or similar activity as LOXL as defined herein.

The active principles are considered as being effective which enable performing an increase of about 2 times of the expression and/or of the activity of LOX, and in parallel performing a decrease of about 0.5 times of the expression of the mRNA of the elastin (Eln), on a model, which comprises at least one cell type which presents an expression and/or an activity of LOX, upon contact of these active principles, with respect to the level of expression and/or of activity of LOX in a control model (without placing the active principles in contact).

It is such that the present invention relates, according to a first aspect, to a screening method of a substance which promotes the activity and/or the formation of LOX or of an homologous or essentially homologous form thereof, for regulating elastogenesis in cases of abnormal or pathological elastogenesis, characterized in that it comprises

placing a potentially active substance in contact with at least one type of cells, which are preferably living, and which are capable of expressing LOX or of an homologous or essentially homologous form thereof, and

analyzing the expression of LOX or of an homologous or essentially homologous form thereof, notably with the aim of identifying whether said potentially active substance stimulates the expression of LOX or of an homologous or essentially homologous form thereof.

Advantageously, the method comprises placing said potentially active substance in contact with at least one type of cells which are capable of expressing the protein elastin, it being possible for these cells to be the same as those expressing LOX, and comprises analyzing the expression of the protein elastin, notably with the aim of identifying whether said potentially active substance promotes the reduction or the inhibition of the synthesis of elastin.

Advantageously, it is sought whether said active substance:

stimulates the expression of at least one part of a sequence of nucleotides encoding the protein LOX, and/or

stimulates the expression of a sequence of peptides which is present within the structure of the protein LOX; and

inhibits the expression of at least one part of a sequence of nucleotides encoding the protein elastin, and/or

inhibits the expression of a sequence of peptides which is present within the structure of the protein elastin.

Advantageously, analyzing the expression of LOX and/or of the protein elastin is carried out by qualitative and/or quantitative analysis of the expression of a sequence of nucleotides encoding LOX and/or encoding tropoelastin.

Advantageously, analyzing the expression of LOX and/or of the protein elastin makes use of a reverse transcription polymerase chain reaction (RT-PCR) which comprises the use of primers which hybridize with at least one part of the sequence of nucleotides of the complementary DNA encoding LOX (SEQ ID No 2), in order to amplify at least one part of the sequence of nucleotides encoding the LOX, and/or the use of primers which hybridize with at least one part of the sequence of nucleotides of the complementary DNA encoding tropoelastin (SEQ ID No 5), in order to amplify at least one part of the sequence of nucleotides encoding tropoelastin.

Advantageously, the method also comprises a step of locating the expression of LOX which is carried out on a reconstructed skin model or on a biopsy:

by in situ hybridization, notably of at least one part of a sequence of nucleotides encoding LOX, in particular by using at least one DNA probe which hybridizes with at least one part of the sequence of nucleotides encoding LOX, and/or notably of at least one part of a sequence of nucleotides encoding tropoelastin, for example with a DNA probe which hybridizes with at least one part of the sequence of nucleotides encoding tropoelastin, and/or

by immuno-detection, notably of at least one part of a sequence of peptides encoding LOX, in particular by using at least one antibody which is capable of recognizing all or part of LOX, and/or at least one antibody which is capable of recognizing all or part of the elastin.

Advantageously, the step of immuno-detection or of in situ hybridization enables performing the traceability of the LOX and of the elastin, notably in the epithelial tissues and/or in the connective tissues, said tissue originating from at least one reconstructed skin model or from biopsies.

Advantageously, the screening method comprises the comparison of the expression of LOX with the expression of LOX expressed in a control which does not comprise said active substance, and/or the comparison of the expression of the protein elastin with the expression of the protein elastin expressed in a control which does not comprise said active substance.

Advantageously, the cells comprise fibroblasts, originating notably from normal human skin, such as, for example, originating from the foreskin or from a skin of an adult subject.

Advantageously, the cells comprise epithelial cells such as keratinocytes, originating notably from normal human skin, such as, for example, originating from the foreskin or from a skin of an adult subject.

Advantageously, the living cells originate from at least one skin having a particular location, for example from the face, from the abdomen, or from the breasts, and being able to be characterized as being <<photo-aged >> or as being <<punctually exposed >> to sun's radiation or not.

Advantageously, the screening method makes use of a reconstructed skin model, preferably at least one dermis model which comprises fibroblasts, or a biopsy-based model.

Advantageously, the screening method makes use of a reconstructed skin model, preferably at least one epidermis model which comprises keratinocytes.

The skin model used is advantageously the Mimeskin® reconstructed skin model but may also be a model of connective matrix, of epidermis or of epithelium, or of reconstructed skin or mucous membrane:

1) The three-dimensional connective matrix (dermis or chorion) culture model, comprises a support which is sown with stromal cells in order to form reconstructed dermis or reconstructed chorions. This support is preferably selected from

an inert support selected from the group consisting of a semi-permeable synthetic membrane, in particular a semi-permeable nitrocellulose membrane, a semi-permeable nylon membrane, a Teflon membrane or a Teflon sponge, a semi-permeable membrane of polycarbonate or polyethylene, polypropylene, or of polyethylene terephthalate (PET), a semi-permeable Anopore inorganic membrane, of cellulose acetate or cellulose ester (HATF) membrane, a semi-permeable Biopore-CM membrane, a semi-permeable polyester membrane, a membrane or a film of polyglycolic acid. In this group, the dermal models Skin²™ model ZK1100 and Dermagraft® and Transcyte® (Advanced Tissue Sciences) for example, are found;

a cell culture-treated plastic (formation of a dermal leaf: Michel M. et al. in In Vitro Cell. Dev Biol.-Animal (1999) 35: 318-326);

a gel or a membrane based on hyaluronic acid (Hyalograft® 3D—Fidia Advanced Biopolymers) and/or on collagen and/or on fibronectin and/or on fibrin; in this group, dermal model Vitrix® (Organogenesis) for example is found;

a porous matrix, which is surfaced or non-surfaced, made from collagen being able to contain one or more glycosaminoglycans and/or eventually chitosan (EP 0 296 078 A1 of the CNRS, WO 01/911821 and WO 01/92322 of Coletica).

2) The three-dimensional epidermis or epithelium culture model Comprises a support which is sown or not beforehand with stromal cells, particularly fibroblasts, and then with epithelial cells and in particular keratinocytes, so as to obtain reconstructed epithelia or epidermis. This support is preferably selected from:

an inert support selected from the group consisting of a semi-permeable synthetic membrane, in particular a semi-permeable nitrocellulose membrane, a semi-permeable nylon membrane, a Teflon membrane or a Teflon sponge, a semi-permeable membrane of polycarbonate or polyethylene, polypropylene, or of polyethylene terephthalate (PET), a semi-permeable Anopore inorganic membrane, of cellulose acetate or cellulose ester (HATF) membrane, a semi-permeable Biopore-CM membrane, a semi-permeable polyester membrane; in this group, the reconstructed models Epiderm and Epithelia (Skinethic®) are found, as well as the models EpiDerm®, EpiAirway®, EpiOccular® (Mattek Corporation);

a film or a membrane based on hyaluronic acid and/or on collagen and/or on fibronectin and/or on fibrin. In this group, the models: Episkin® (L'Oreal) and Laserskin® (Fidia advanced Biopolymers), in particular, can be cited.

3) The Three-Dimensional Reconstructed Skin or Mucous Membrane Culture Model

Comprises a matrix support (dermal or of chorion) which is sown with epithelial cells so as to obtain a reconstructed skin. This support is preferably selected from:

an inert support selected from the group consisting of a semi-permeable synthetic membrane, in particular a semi-permeable nitrocellulose membrane, a semi-permeable nylon membrane, a Teflon membrane or a Teflon sponge, a semi-permeable membrane of polycarbonate or polyethylene, polypropylene, or of polyethylene terephthalate (PET), a semi-permeable Anopore inorganic membrane, of cellulose acetate or cellulose ester (HATF) membrane, a semi-permeable Biopore-CM membrane, a semi-permeable polyester membrane, said inert support containing stromal cells, in particular fibroblasts,

a gel based on collagen and/or hyaluronic acid and/or fibronectin, and/or on fibrin comprising stromal cells, in particular fibroblasts,

a porous matrix, which is surfaced or non-surfaced, made from collagen being able to contain one or more glycosaminoglycans and/or eventually chitosan, these porous matrices integrating stromal cells, in particular fibroblasts,

a human or animal de-epidermisised dermis or dead dermis.

In this group, the models Apligraf® (Organogenesis), ATS-2000 (CellSystems® Biotechnologie Vertrieb), as well as Skin²™ (ZK1200-1300-2000—Advanced Tissue Science), in particular, can be cited.

Furthermore, models exist which are dedicated to tissue therapy and which can also be the subject of such studies. The models Epidex™ (Modex Therapeutiques), Epibase® (Laboratoire Genevrier), Epicell™ (Genzyme), Autoderm™ and Transderm™ (Innogenetics), can be cited.

Advantageously, said active substance is selected from the group consisting of plant or synthetic polyphenols, an extract of galega or of cardamon, a saturated fatty acid, a polyol such as xylitol, adragant gum, an extract of cereals such as barley or oats.

According to a second aspect, the invention relates to the use of the LOX having the Sequence ID No 1 or of an homologous or essentially homologous form thereof or of a first substance which promotes the activity and/or the formation of LOX combined with a second substance which promotes the reduction or the inhibition of the synthesis of elastin, for the manufacture of a composition which is intended notably for regulating elastogenesis in cases of abnormal or pathological elastogenesis. This composition is advantageously intended to stimulate the enzymatic activity or the expression of the isoform of lysyl oxidase LOX or of an homologous or essentially homologous form thereof and to inhibit the synthesis of the protein elastin.

Advantageously, the abnormal or pathological elastogenesis is in particular encountered in cases of fibrosis, of solar elastosis, of stretch marks, of dystrophic scars, of eczemas and/or a stromal reaction to cancers which are associated with a disorganized and/or non-functional elastic tissue.

Advantageously, the first substance and the second substance are identical.

Advantageously, said second substance promotes the reduction or the inhibition of the synthesis of tropoelastin having the sequence ID No 3 or of an homologous or essentially homologous form thereof, the precursor protein of elastin.

Advantageously, said second substance:

i) comprises a region which fixes to at least one part of the sequence of nucleotides of the human elastin gene promoter (Pr) (SEQ ID No4) or of an homologous or essentially homologous form thereof or,

ii) modulates the expression of a protein which fixes to at least one part of the sequence of nucleotides of the human elastin gene promoter (Pr) (SEQ ID No4) or of an homologous or essentially homologous form thereof, so as to reduce or to inhibit the synthesis of tropoelastin.

Advantageously, said active substance is selected from the group consisting of plant or synthetic polyphenols, an extract of galega or of cardamon, a saturated fatty acid, a polyol such as xylitol, gum adragant, an extract of cereals such as barley or oats.

According to a third aspect, the invention relates to a cosmetic composition characterized in that it comprises an active substance as defined above, optionally in a mixture with a cosmetically acceptable excipient.

According to a fourth aspect, the invention relates to a neutraceutical composition characterized in that it comprises an active substance as defined above, optionally in a mixture with an excipient acceptable for food.

According to a fifth aspect, the invention relates to a pharmaceutical composition characterized in that it comprises an active substance as defined above, optionally in a mixture with a pharmaceutically acceptable excipient.

The active principle, or the combination of active principles, or the combination of the enzyme LOX, or of a derivative form, with an active principle which inhibits the synthesis of the protein elastin, are advantageously in a mixture with an excipient which is acceptable by the human body. For example the excipient contains at least one compound selected from the group consisting of preservatives, emollients, emulsifiers, surfactants, moisturisers, thickeners, conditioners, matifying agents, stabilizers, antioxidants, texture agents, brightening agents, filmogenic agents, solubilisers, pigments, dyes, perfumes and solar filters. These excipients are preferably selected from the group consisting of amino acids and their derivatives, polyglycerols, esters, polymers and derivatives of cellulose, lanolin derivatives, phospholipids, lactoferrins, lactoperoxidases, sucrose-based stabilizers, E vitamins and its derivatives, natural and synthetic waxes, plant oils, triglycerides, insaponiflables, phytosterols, plant esters, silicones and its derivatives, protein hydrolysates, jojoba oil and its derivatives, lipo/hydrosoluble esters, betaines, aminoxides, plant extracts, esters of sucrose, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerol, sodium dihydroxycetyl, isopropyl hydroxycetyl ether, glycol stearate, triisononaoine, octyl cocoate, polyacrylamide, isoparaffin, laureth-7, a carbomer, propylene glycol, glycerol, bisabolol, dimethicone, sodium hydroxide, a perfume, PEG 30-dipolyhydroxysterate, capric/caprylic triglycerides, cetearyl octanoate, dibutyl adipate, grape seed oil, jojoba oil, magnesium sulphate, EDTA, cyclomethicone, xanthan gum, citric acid, sodium lauryl sulphate, mineral waxes and oils, isostearyl isostearate, propylene glycol dipelargonate, propylene glycol isostearate, PEG 8 Beeswax, hydrogenated palm tree heart oil glycerides, hydrogenated palm oil glycerides, lanolin oil, sesame oil, cetyl lactate, lanolin alcohol, castor oil, titanium dioxide, colorants, and pigments, lactose, sucrose, low density polyethylene, and an isotonic saline solution.

Advantageously, the compositions cited above are formulated in a form selected from the group consisting of a solution, which is aqueous or oily, an aqueous cream or gel or an oily gel, notably in a pot or in a tube, notably a shower gel, a shampoo; a milk; an emulsion, a microemulsion or a nanoemulsion, notably an oil-in-water or water-in-oil or multiple or silicone-containing microemulsion or nanoemulsion; a lotion, notably in a glass bottle, a plastic bottle or in a measure bottle or in an aerosol; an ampoule; a liquid soap; a dermatological bar; an ointment; a foam; an anhydrous product, preferably a liquid, pasty or solid anhydrous product, e.g. in the form of a stick, notably in the form of a lipstick.

Advantageously, the compositions which are sufficiently liquid can be administered, notably via the parenteral, ocular, pulmonary, oral or nasal route.

Advantageously, the pasty or dry compositions (pastes, powders, tablets, capsules, granules, suppositories.), can be introduced into the body notably via the oral, sublingual, nasal or rectal route.

Advantageously, when the formulation of the composition allows it, the administration route is cutaneous or transmucosal, notably by application of the composition on the skin or on a mucous membrane.

Advantageously, from the various formulations and route of administration, the person skilled in the art will select the one which is adequate for the effectiveness sought after.

According to a sixth aspect, the invention relates to a method of cosmetic care which comprises applying an effective amount of at least one active principle, or of one of the types of combinations, as defined above, or of a composition as defined above, notably in cases of solar elastosis, stretch marks and/or dystrophic scars.

According to a seventh aspect the invention relates to a method of locating the expression of LOX or of an homologous or essentially homologous form thereof, and optionally of the protein elastin in tissues with the aim of performing the traceability of neo-collagenogenesis and/or of neo-elastogenesis, notably in epithelial tissues and/or in connective tissues, said tissues originating from at least one reconstructed skin model or a biopsy-based model, characterized in that the method comprises a step of immuno-detection of the protein LOX and optionally of the protein elastin, for example by specific antibodies.

The invention also relates to a method of locating the expression of the isoform of lysyl oxidase LOX or of an homologous or essentially homologous form thereof, which comprises a step of immuno-detection or of in situ hybridization, notably with the aim of performing the traceability of neo-elastogenesis, notably in the epithelial tissues and/or in the connective tissues.

The invention also relates to the treatment of a deficiency of the enzymatic activity of the isoform of the protein lysyl oxidase LOX or of an homologous or essentially homologous form thereof which comprises administering to a subject a therapeutically effective amount of a composition which comprises an active principle, or of one of the types of combinations, as defined above, which increases the enzymatic activity of the protein lysyl oxidase LOX or of an homologous or essentially homologous form thereof and which inhibits the synthesis of the protein elastin.

Advantageously this method of treatment is used for regulating elastogenesis in cases of pathological, disorganized and/or non-functional elastogenesis, as in cases of fibrosis, of solar elastosis, of stretch marks, of dystrophic scars, of eczemas and/or a stromal reaction to cancers which are associated with a disorganized and/or non-functional elastic tissue.

### DETAILED DESCRIPTION OF THE INVENTION

The inventors have unexpectedly demonstrated that the isoform LOX of lysyl oxidase was not stimulated enough locally in order to regulate elastogenesis in cases of pathological, disorganized and/or non-functional elastogenesis, such as in cases of solar elastosis, of stretch marks, and/or of dystrophic scars. The inventors have unexpectedly demonstrated that in cases of pathological, disorganized and/or non-functional elastogenesis, as in cases of solar elastosis, of stretch marks, and/or of dystrophic scars, notably for protecting the skin from ageing, from UVs, or in cases of eczema and other comparable dysfunctions, it is necessary to be able to re-induce the expression of LOX in reducing the synthesis of elastin, which has a tendency to be over-produced and deposited in a non-functional way in the cases cited above.

The inventors have in fact demonstrated that this isoform of the family of lysyl oxidases (LO) is associated with elastogenesis in a reconstructed skin model producing elastic fibers. In seeking whether this isoform was present or absent in the skin of various ages and during skin alterations, the inventors noticed the simultaneous presence or absence of this isoform and of elastogenesis, and this enables indicating that the reactivation of the expression of this isoform of LO (LOX) as well as the inhibition of the synthesis of elastin, enables regulating elastogenesis in cases of pathological, disorganized and/or non-functional elastogenesis, as in cases of fibrosis, of solar elastosis, of stretch marks, and/or of dystrophic scars.

The inventors have thus developed a method which enables visualizing increased expressions of this isoform of LOX, and then have sought active principles, notably amongst plant extracts or chemical molecules, which in particular stimulate the expression of the mRNAs encoding LOX, and actives which be capable of inhibiting, simultaneously or not, the expression of the protein elastin. The actives selected, or a combination of the enzyme LOX with an active principle which inhibits the expression of the protein elastin were then incorporated in cosmetic and dermo-pharmaceutical formulations, for applications in reducing pathological, disorganized and/or non-functional elastogenesis.

The inventors have developed specific antibodies of the mature LOX and LOXL forms (vide Examples 1 and 2), and have demonstrated in this way that the absence of the isoform of lysyl oxidase LOX is mainly responsible for problems of formation of collagen fibers, notably during pathological, disorganized and/or non-functional elastogenesis.

The isoforms LOXL2, LOXL3 and LOXL4 are not or are little expressed in the dermis and are not involved in elastogenesis (vide Example 4). LOX is the enzyme which is responsible for the maturation of collagen (by cross-linking), and this explains that this enzyme might be involved in densifying the stroma.

Within the context of the present invention, the inventors have implemented a method of locating the expression of LOX. Notably, this method of location comprises the immuno-detection of LOX. The expression of the protein elastin can also be demonstrated by this method. It has been demonstrated by the inventors' studies that LOX is associated with microfibrils and with the formation of collagen (FIG. 5). Elastin was detected in the same dense deposits and in the microfibrils. This detection is made on reconstructed skin models, and notably on reconstructed skin models, observed 30 days after the deposit of the keratinocytes. The association of LOX with the microfibrils and with the collagen fibers was also confirmed in the skin of the foreskin, notably by electron microscopy after immuno-detection. LOX is expressed in the dermis of the skin of foreskin taken from young patients (a few months), which have still a large synthesis of elastin. LOX is always expressed in the adults dermis, notably in the dermis of the skin of the neck, the breast, the abdomen or the face. A high expression was also observed of LOX in the epidermis of human skin, with however a late extinction of the expression of this enzyme when the human skins originate from aged subjects (91 years old) (vide Example 6).

With regard to scars, LOX was not observed in these zones, neither three months after the scar, nor five years after the formation of the scar. In this context, it is to be noted that the elastin which is expressed at three months is no longer present on this scar tissue five years after the formation of the scar.

The inventors have thus demonstrated the role of LOX in the formation of collagen fibers and of elastic fibers, notably by using reconstructed skin models or dermis of the foreskin of young patients.

The inventors have also demonstrated the deficit of expression of LOX in the scar tissue zones.

The whole of the inventors' studies has enabled developing a screening method of an active principle which regulates elastogenesis in cases of pathological, disorganized and/or non-functional elastogenesis, such as solar elastosis. This regulation manifests itself in particular by the cross-linking of the collagen and the elastin.

Within the context of the present invention, the inventors have implemented a method of in situ hybridization which thus enables locating and verifying the presence of the expression of messenger RNAs which encode LOX in particular. This in situ hybridization is notably carried out by double strand DNA probes which are labelled with digoxigenin on sections of 35 day reconstructed skin models, included in paraffin. This in situ hybridization was also carried out in order to verify the expression of the messenger RNAs of tropoelastin and of collagen Ial (vide Example 7).

The expression of mRNAs of LOX is observed throughout the whole dermis. In the epidermis, the cells of the basal layer do not express or express little LOX, whereas the cells of the spinous and granular layers strongly express it. The expression of the mRNA of collagen Iα1 is localized in the dermis but not in the epidermis.

Therefore, the aim of this is to be applied to the verification of the stimulation of the mRNAs of LOX and eventually of the inhibition of the mRNAs of elastin in a reconstructed skin model, e.g. after application of an active principle which eventually regulates the elastogenesis in cases of pathological, disorganized and/or non-functional elastogenesis, such as fibrosis or solar elastosis.

Within the context of the present invention, the hLOX gene was activated by the addition of keratinocytes in a reconstructed skin model, and notably in reconstructed skin model Mimeski®, (Coletica, Lyons, France). The induction of the synthesis of mRNAs of LOX is observed simultaneously to that of the mRNAs of collagen 1 alpha1 (COLL alpha1), and notably appears about 6 days after the addition of the keratinocytes on the dermis equivalent.

The expression of the genes of interest encoding LOX, encoding elastin, encoding LOXL, as well as the one encoding actin, was analyzed by real time RT-PCR. This technique enables precisely quantifying the expression of a gene in comparing it to that of actin which is considered to be constant. The regulation of the level of expression of these genes can therefore be quantified, and in particular the expression of the gene encoding LOX and the one encoding elastin.

The synthesis of LOX mRNA decreases on average by 40% in the fibroblasts originating from aged donors with respect to the fibroblasts which originate from foreskin (vide Example 8). The present invention provides a method which enables quantifying the expression of LOX, notably in fibroblasts. The present invention aims to implement these various techniques so as to identify active principles which enable regulating elastogenesis in cases of pathological, disorganized and/or non-functional elastogenesis, such as fibrosis or solar elastosis.

In general, the methods of the present invention implement seeking the expression of the protein LOX and of elastin, and notably seeking the expression of the messenger RNAs encoding LOX and those encoding elastin (vide Example 9).

The invention also relates to the active principles which stimulate the expression of LOX and which inhibit the expression of the protein elastin for regulating elastogenesis in cases of pathological, disorganized and/or non-functional elastogenesis, such as fibrosis or solar elastosis (vide Example 9 to 11). The invention also relates to the use of these active principles for preparing cosmetic or pharmaceutical compositions (vide Examples 12 to 18).

The variations of expression of LOX and of elastin can be observed in the gene, of the messenger RNA, or of the protein, directly. These variations enable regulating elastogenesis in cases of pathological, disorganized and/or non-functional elastogenesis, such as fibrosis or solar elastosis, notably by virtue of the cross-linking of the collagen by the enzyme LOX.

Other aims, features and advantages of the invention will appear clearly to the person skilled in the art upon reading the explanatory description which makes reference to the following Examples.

The Examples make up an integral part of the present invention and any feature appearing novel with respect to any prior state of the art from the description taken in its entirety, including the Examples, makes up an integral part of the invention in its function and in its generality.

Thus, every Example is of general scope.

Furthermore, in the Examples, all the percentages are given by weight, unless indicated otherwise, the temperature is expressed in degrees Celsius unless indicated otherwise, and the pressure is atmospheric pressure, unless indicated otherwise.

### EXAMPLES

### Example 1

### Preparation of Specific Antibodies of the Mature Forms of LOX and LOXL

The invention has first of all covered the development of novel specific antibodies of the mature forms of LOX and LOXL. The antibodies were developed against the mature regions of LOX and LOXL. The antigenic regions were selected in order to present the minimum of similarity with the corresponding regions on the other isoforms of lysyl oxidases (LOs). The antibodies obtained against the regions of the peptides LOX^{V228-S280} were called anti-LOXmat and similarly for the antibodies obtained against the region of the peptides LOXL^{R231-G368} called anti-LOXLpro.

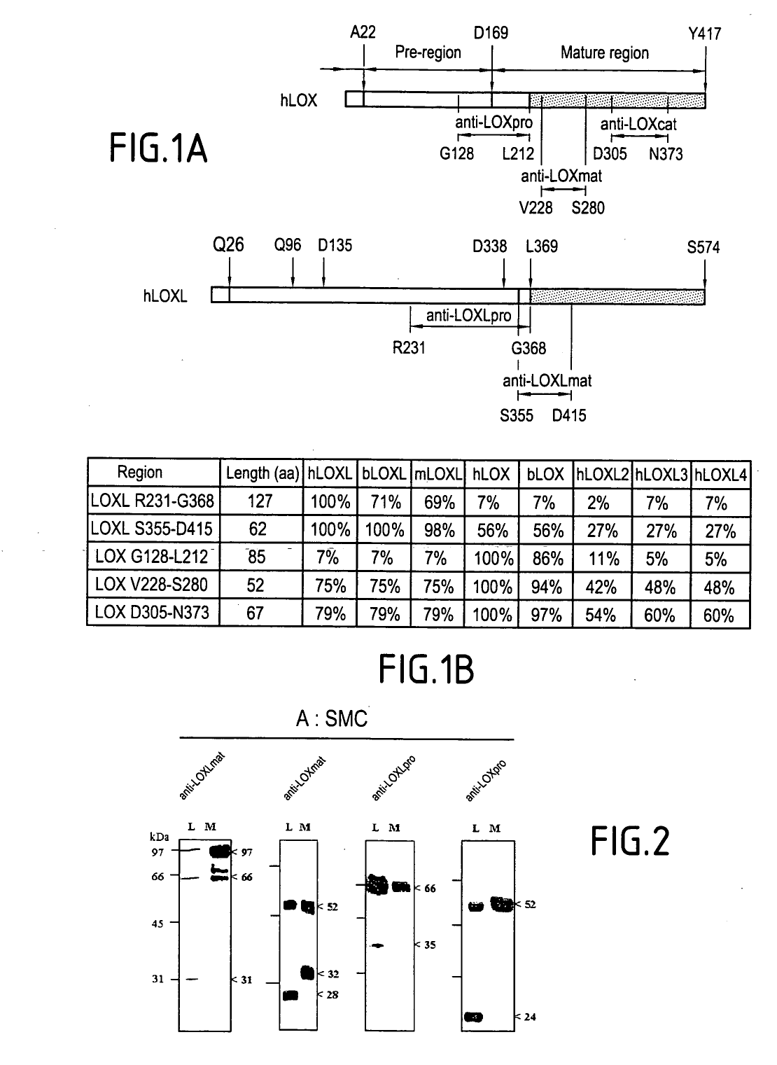

In FIG. 1: description of the sequences of the LOs defined for giving the specific antibodies: This figure represents the steps which have led to the selection of the antigenic regions in order to develop the anti-LOX and anti-LOXL antibodies.

FIG. 1(A): Schematic representation of hLOX (human LOX protein) and hLOXL (human LOXL protein).

The sequences of hLOX and hLOXL are indicated with open boxes, dotted in the C-terminal regions, in order to highlight the regions of high similarity. The position of the cleavage of the pre-region and of the cleavage sites by procollagen-C-proteinase (PCP), on the A22 and D169 residues of hLOX respectively, was indicated. The position of the cleavage of the pre-region of LOXL, before the Q26 residue, of the N-terminal maturation site of the 56 kDa precursor (before D135), and the position of the cleavage sites by PCP of the LOXL precursor of 56 kDa (before the D 338 residues), are indicated. The corresponding LOXL proteins Q²⁶-S⁵⁷⁴, D¹³⁵-S⁵⁷⁴, and D³³⁸-S⁵⁷⁴ would display a deduced molecular mass of approximately 63 kDa, 54.6 kDa, and 26.7 kDa, respectively. The location of the recombinant peptides used for obtaining the anti-LOX antibodies were indicated the G128-L212 peptide for the anti-LOXpro, the V228-S280 peptide for the anti-LOXmat, and the D305-N373 peptide for the anti-LOXcat. The location of the recombinant peptides used for developing the anti-LOXL antibodies were indicated: the R231-G368 peptide for the anti-LOXLpro and S355-D415 for the anti-LOXLmat.

FIG. 1(B): The percentage of similarity between the antigenic regions of LOX and LOXL with their equivalents on the LO isoforms, was indicated in this Table.

In the Table of FIG. 1(B), hLOXL represents the human LOXL protein, bLOXL represents the bovine LOXL protein, mLOXL represents the mouse LOXL protein, hLOX represent the human LOX protein, bLOX represents the bovine LOX protein, hLOXL2 represents the human LOXL2 protein, hLOXL3 represents the human LOXL3 protein, hLOXL4 represents the human LOXL4 protein.

The length column (aa) contains the value of the number of amino acids contained in the corresponding regions. In order to obtain the antibodies, the chimeric genes ere constructed by inserting the defined sequence of hLOXL or hLOX in phase with the gene of glutathion-S-transferase (GST), in the BamHI-XhoI sites of the expression plasmid pGEX-4T-3 (Amersham Biosciences).

The fusion gene GST-LOXL^{S355-D415} was constructed by introducing the cDNA sequence of HLOXL (hLOXL cDNA), produced by PCR with the sense primer 5′-TTGGATCCAGCGTAGGCAGCGTGTAC-3′ (SEQ ID No16), and antisense primer 5′-AAACTCGAGCATCGTAGTCGGTGGC-3′-(SEQ ID No17). The fusion gene GST-LOX^{G128-L212} was constructed by introducing hLOX cDNA amplified with sense primer 5′-TCGGATCCGGCTACTCGACATCTAGAGCC-3′ (SEQ ID No18) and antisense primer 5′-GTCCTCGAGACCGTACTGGMGTAGCC-3′ (SEQ ID No19), respectively. The fusion gene GST-LOX^{V228-S279} was constructed by introducing the hLOX sequence amplified with sense primer 5′-TTGGATCCGTGCAGMGATGTCCATGTAC-3′ (SEQ ID No20) and antisense primer 5′-TTTCTCGAGGCTGGGTMGAAATCTGATG-3′ (SEQ ID No21), respectively. The fusion gene GST-LOX^{D306-N373} was constructed by introducing hLOX cDNA amplified with sense primer 5′-CACTATGGATCCCTTGATGCCAACACCC-3′ (SEQ ID No22) and antisense primer 5′-CACGACCTTTAGGATATCGTTTCCAGG-3′ (SEQ ID No23), respectively. For the whole of these amplifications by PCR, the Taq polymerase High Fidelity (Roche Diagnostic, Meyman, France) was used.

The fusion proteins GST-LOX and GST-LOXL, as well as the rabbit polyclonal antibodies, were obtained and purified as described above for the fusion proteins originating from the expression of the fusion genes GST-LOXL^{S355-D415} and GST LOX^{G128-L212} (Borel et al, J. Biol. Chem, 276: 48944-49, 2001; Decitre et al, Lab. Invest., 78, 143-151, 1998). For the adsorption experiments, the antibodies were incubated for 3 hours at 20° C. with the fusion proteins, themselves adsorbed on a nitrocellulose membrane, like Hybond-ECL membrane (Amersham Biosciences) before the immuno-detection.

These pieces of work have first of all enabled demonstrating the mature forms of LOX and LOXL, by virtue of the immunochemical and biochemical characterization of the mature proteins (vide Example 2, FIG. 2). The antibodies developed are distinguished from those used in the prior art for LOXL, which do not enable a recognition of the mature form of LOXL (Decitre et al., Lab Invest 78: 143-151, 1998). The invention consisted in using the anti-LOXLmat antibody and anti-LOXmat, and this enabled demonstrating a protein of 31 kDa, which is recognized by the anti-LOXLmat but not by the anti-LOXmat, and which corresponds-to the mature-form of LOXL. This part of the invention demonstrates a real progress over the prior art, notably with reference to the patent of Csiszar et al., which describes all the proteins originating from the genes of the LO family without defining the features of them (WO 01/83702 A2 patent: Novel members of the lysyl oxidase family of amine oxidases related applications).

### Example 2

### Immuno-Detection of LOX and LOXL of Muscle Cells by Virtue of the Novel Antibodies Anti LOX and Anti-LOXL

FIG. 2 represents photographs of electrophoreses which were carried out as indicated below. These electrophoreses demonstrate the characterization of the mature proteins of LOX and LOXL, of smooth muscle cells (SMC) by virtue of the antibodies anti-LOX and anti-LOXL, which is identified in Example 1.

The proteins of the cell strain (L) and of the cell culture medium (M) of a cell line of rat smooth muscle (developed by Jean-Marie Daniel Lamaziere, Bordeaux) were extracted and detected by western blotting by using the antibodies anti-LOXLmat, anti-LOXmat, anti-LOXLpro and anti-LOXpro. The cells were cultivated at 37° C. in an atmosphere of 5% CO₂ in DMEM medium (Sigma) containing 10% fœtal calf serum, 2 mM glutamine and 50 μg/ml gentamycin. The cell strain proteins, which are washed twice with PBS buffer, were extracted for 2 hours at 4° C. with slow agitation in the lysis buffer (16 mM phosphate buffer pH 8, 0.5% NP40, protease inhibitors (Complete Mini, Roche Diagnostics), and urea 6 M). The lysates were diluted with two volumes of 16 mM phosphate buffer pH 8, with protease inhibitors (Complete Mini, Roche Diagnostics, Meylan, France), and centrifuged for 20 minutes at 15,000 g. The soluble proteins were precipitated by adding 10% trichloroacetic acid (TCA) before the electrophoresis. The proteins of the culture media were recovered from medium of cells cultivated for 48 hours without serum, and precipitated by adding 10% TCA or 50% saturated ammonium sulphate.

For the immuno-detection, the proteins are separated by 10% SDS-polyacrylamide gel electrophoresis. The proteins were transferred onto a polyvinylidene fluoride (PVDF) membrane (Immobilon p^{SQ}, Millipore) and were immuno-detected as described above (Borel et al., J. Biol. Chem, 276: 48944-49, 2001).

The developed antibodies thus enable characterizing and locating the mature and immature forms of LOX and LOXL in the biological tissues.

### Example 3

### Demonstration of the Role of LOX in Elastogenesis

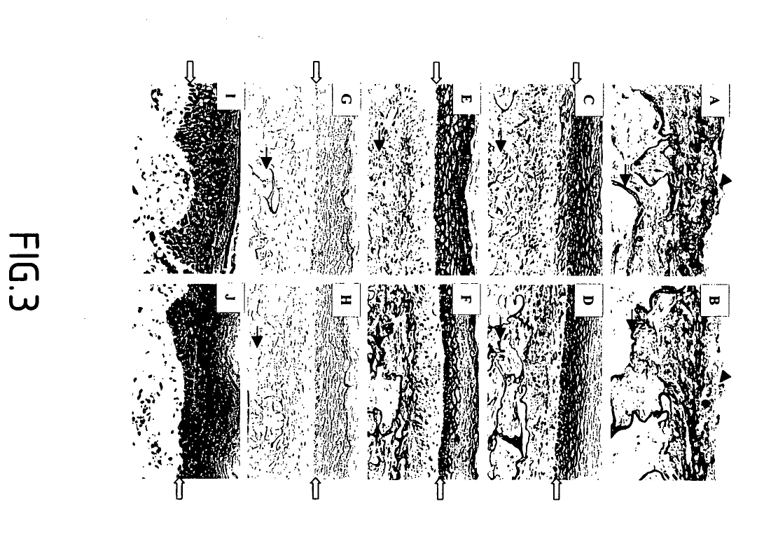

The inventors have demonstrated that the LOX and LOXL proteins can be associated with the formation of connective tissue in the dermis of reconstructed skin models by immuno-histochemistry (FIG. 3). This demonstration was obtained without any ambiguity by virtue of the use of anti-LOX and anti-LOXL antibody couples, directed against the pro-enzymatic regions and mature regions of the two enzymes (LOX and LOXL).

On FIG. 3 representation is made of the immuno-histological detection of LOXL and LOX in the reconstructed skin (RS) and the normal human skin:

The immuno-detection of LOXL (A, C, E, G) of reconstructed skins at days 16 (A), 35 (C), and 45 (E), by using anti-LOXL^{R231-G368} (A, C, E) or anti-LOXL^{R231-G368} adsorbed with the corresponding peptide GST-LOXL^{R231-G368} before the immuno-detection (G).

The immuno-detection of LOX (B, D, F, H) of reconstructed skins at days 16 (B), 35 (D), and 45 (F), by using anti-LOX^{V228-S279} (B, D, F) or anti-LOX^{V228-S279} adsorbed with the corresponding peptide GST-LOX^{V228-S279} before the immuno-detection (H).

The immuno-detection of LOXL (I) and of LOX (J) in the skin of human foreskin, by using anti-LOXL^{R231-G361} (I) and anti-LOX^{V228-S279} (J). The position of the dermal-epidermal junction is indicated with an open arrow, that of the dermal substrate with an arrow, and the location of the keratinocytes at day 16 is indicated with an arrow head.

The reconstructed skin (Mimeskin®, Coletica, Lyons, France) was prepared in Bouin's fixative (LOX, LOXL, elastin) or in a 10% formol solution (for the elastin), and then included in paraffin. 6 μm thick sections were ridded of paraffin and were whitened in glycine-HCl (100 mmol/l). The anti-LOX and anti-LOXL antibodies are described above.

The antibodies were used at the following dilution: 1:500 (anti-LOXLR²³¹-G³⁶⁸), 1:100 (anti-LOX^{V228-S279}, anti-LOXLS^{355-D}⁴¹⁶). The immune complexes were detected with a rabbit (goat) anti IgG conjugated with peroxidase (DAKO, Trappes, France), by using diaminobenzidine as substrate (DAKO).

LOX and LOXL are thus candidates for participating in elastogenesis in a reconstructed skin model notably such as Mimeskin®.

### Example 4

### Demonstration of the Role of LOXL2, LOXL3, and LOXL4 in Elastogenesis

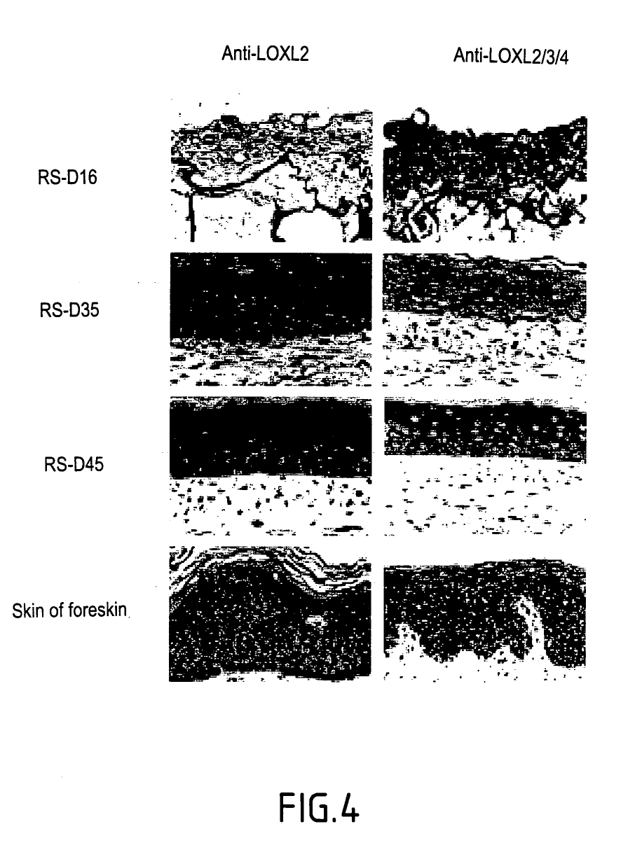

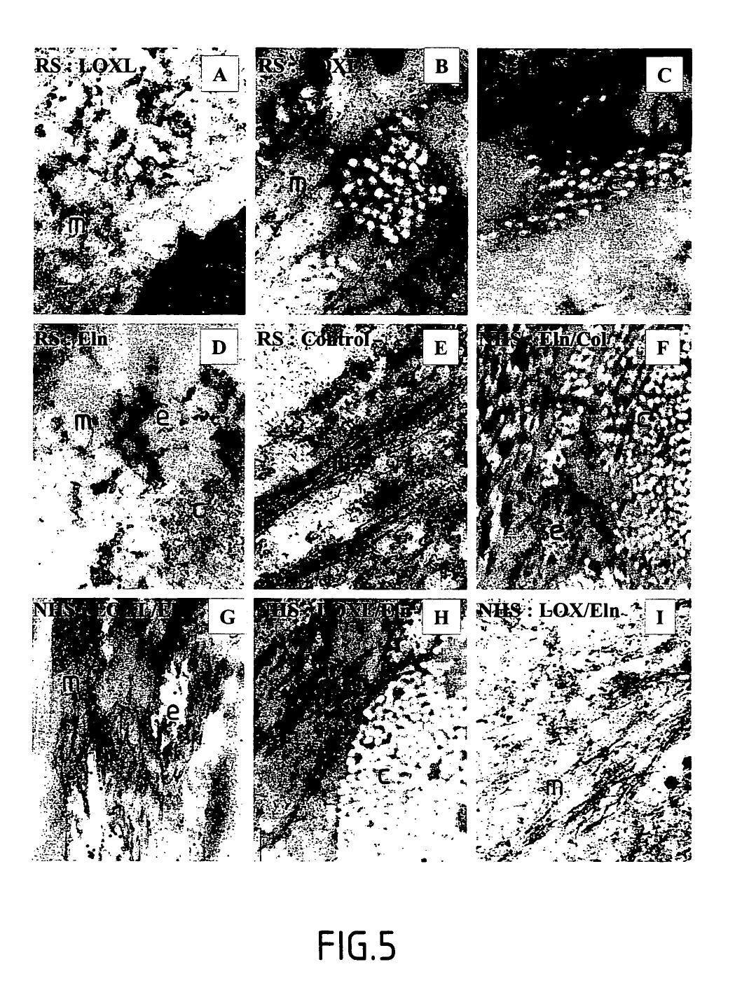

The invention has also borne upon the development of two novel anti-LOXL2 antibodies, one of these antibodies theoretically also recognizing LOXL3 and LOXL4. This has enabled defining whether these enzymes are expressed with elastin in the dermis of a reconstructed skin model. The analysis by immuno-histochemistry by using the two anti-LOXL2 antibodies does in fact show that this antigen, as well as the two antigenically linked proteins LOXL3 and LOXL4, are not or are little expressed in the dermis, and that therefore, they do not participate in the elastogenesis. On FIG. 4: the immuno-detection is represented on the sections of reconstructed skin (16, 35 and 45 days) and of human foreskin skin with the antibody anti LOXL2 ⁵¹⁷⁻⁵⁸¹ (left column) and the antibody anti-LOXL2 ⁶⁶⁴⁻⁷²⁰ (theoretically recognizing LOXL2, LOXL3, and LOXL4) (right column). The anti-LOXL2 antibodies were obtained against fusion peptides GST-LOXL2, as described above (Decitre et al, Lab. Invest., 78, 143-151, 1998).-The fusion gene GST-LOX2⁵¹⁷⁻⁵⁸¹ was constructed by introducing the sequence 1543 to 1747 of the human LOXL2 gene (hLOXL2) in the plasmid, as described above.

This segment was generated by PCR with the sense primer 5-GAGCTGGGATCCGCGCACTGCC-3′ and antisense primer 5′-GGCTGAGTCGACGAGGCAGTTCTCC-3′.

The fusion gene GST-LOXL2⁶⁶⁴⁻⁷²⁰ was constructed by introducing the corresponding hLOXL2 sequence, by virtue of the sense primer 5′-CACAGGATCCGMGGAGACATCCAGMG-3′ and antisense primer 5′-TTTCTGAGCTCCTGCATTTCATGATG-3′.

The fusion proteins and the anti-rabbit antibodies generated against these proteins were prepared as described above. The antibody against the 517-580 peptide was called anti-LOXL2, since this region is specific of LOXL2.

The antibody against the 664-734 peptide was called anti-LOXL-R (for <<relative to >>), since this region of LOXL2 possesses a high similarity with LOXL3 and LOXL4 (about 74.6% and 60.5%, respectively).

The reconstructed skins (Mimeskin®, Coletica, Lyons, France) at 16 days (RS-D16), 35 days (RS-D35) and 45 days.(RS-D45), and the human foreskin skin analysed as above by immunohistochemistry with the anti-LOXL2-R and anti-LOXL2 antibodies. Anti-LOXL2 shows an expression of LOXL2 in the epidermis and not in the dermis. While the antibody directed against the common C-terminal region of LOXL2, LOXL3, and LOXL4 confirms the expression of these enzymes in the epidermis and shows a low expression in the dermis but in an zone which does not correspond to the sites of elastogenesis.

LOXL2, LOXL3 and LOXL4 are not involved in elastogenesis.

### Example 5

### Demonstration of the Role of LOX in Elastogenesis

The association between LOXL and LOX on the one hand, and the elastic fibers or the microfibrils on the other, was clearly demonstrated in transmission electron microscopy by the present invention.

LOX and LOXL associated with the microfibrils constitute the framework on which the elastin is deposited, whereas only LOX is associated with the formation of the collagen fibers (vide FIG. 5).

On FIG. 5: The immuno-detection of LOXL, of LOX and of elastin is represented by transmission electron microscopy in the dermal part of the reconstructed skin at 45 days and of the normal human skin.

The tissues were fixed for 3 hours at 4° C. with 4% paraformaldehyde in PBS buffer containing 0.1% glutaraldehyde, and were then washed in phosphate buffer containing 0.4M of sucrose cacodylate and 0.2M lysine, dehydrated in solutions of ethanol, and included in LR White (Euromedex, France). The detection was carried out with primary antibodies diluted to 1:50 in Tris-HCl buffer at pH 8.2, to which 1% bovine serum albumin (BSA) is added. The immune complexes are detected with a rabbit anti-IgG antibody conjugated with colloidal gold particles of 10 and 20 nm (Biocell, Tebu, France) diluted to 1:40. The samples were contrasted with ³% uranyl acetate and lead citrate, and were then examined under a JEOL 1200 EX electron microscope. The immuno-detection was carried out on the reconstructed skin (A-D) and on the skin of human foreskin (F-I). On the reconstructed skin, it was carried out with the antibodies: anti-LOXL (A, B), anti-LOX (C), anti-elastin (Elm) (D), and with a negative control without primary antibody in the dermis (control) (E). A double labelling (F-I) was made on the skin of the human foreskin.

References A-D: Immuno-detection of LOXL, LOX and elastin by electron microscopy in the dermal part of the reconstructed skins at 45 days.

Reference E: Positive control with anti-elastin and anti-collagen I antibodies in the dermis of reconstructed skins at 45 days, i.e. 30 days after the addition of keratinocytes.

References F-I: Double immuno-detection of LOXL, LOX, elastin and collagen by electron microscopy in the dermal part of human foreskin.

References G-H: Double-labelling in the dermal part of the human foreskin with the rabbit anti-LOXL antibody (the rabbit anti-IgG is conjugated with 10 nm gold particles) and the murine anti-elastin antibody (the mouse anti-IgG is conjugated with 20 nm gold particles).

Keys in the Figure: m: microfibrils, c: collagen fibers, e: amorphous elastin. Bar of the scale: 500 nm.

LOXL (A-B) is detected in association with the dense deposits or on the microfibrils, but not with the collagen fibers which appear in white on these sections. The labelling of LOX (C) is low, although a few gold particles could be found with the dense deposits, the microfibrils and the collagen. The anti-elastin antibodies detected the same dense deposits and the microfibrils (D). As in the observations on the reconstructed skin models, LOXL is not associated with the collagen fibers, opposite to the LOX which is very present with the collagen fibers and little present on the microfibrils. The LOXL antigens were detected in association with the microfibrils and around the elastic fibers of the skin of human foreskin.

The labeling of LOX was not observed with the elastic fibers, but was associated with the collagen fibers and with the microfibrils of the skin (I).

### Example 6

### Demonstration of the Relationship between the Expression of LOX and Elastogenesis

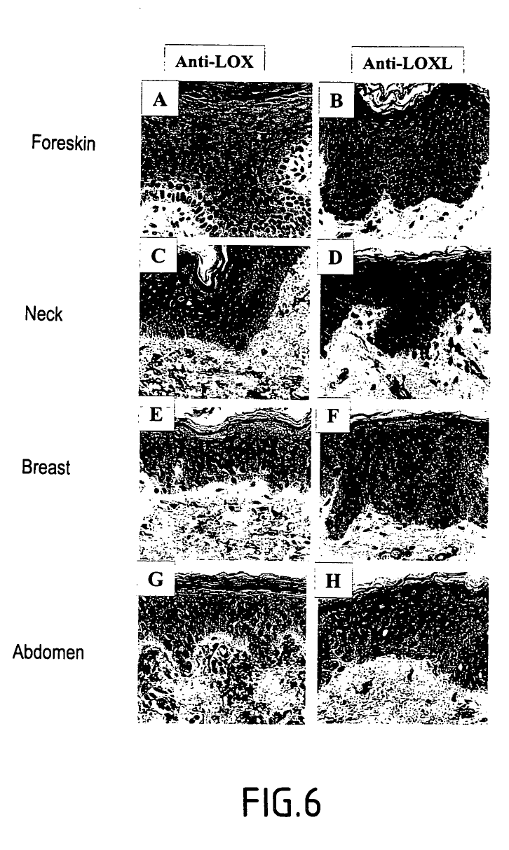

LOXL and LOX are expressed in the dermis of the skin of the foreskin taken from young patients (a few months) still having a high elastin synthesis. LOXL is not expressed in the dermis of the skin of the neck, the breast, the abdomen or of the face, whereas LOX is always expressed in the dermis (FIG. 6).

On FIG. 6: the immuno-detection of LOX— and LOXL is represented in the human skin at various locations.

The antibodies anti-LOX (A, C, E, G) and anti-LOXL (B, D, F, H) were used for detecting the expression of LOX and LOXL in samples of skin of the foreskin (A, B), of the neck (C, D), of the breast (E, F) and of the abdomen (G, H) originating from the tissue bank of the Edouard Herriot Hospital (Lyons, France). The tissues were fixed with Bouin's reagent, included in paraffin, and treated for the immuno-detection as was carried out for the immuno-detections which are described above.

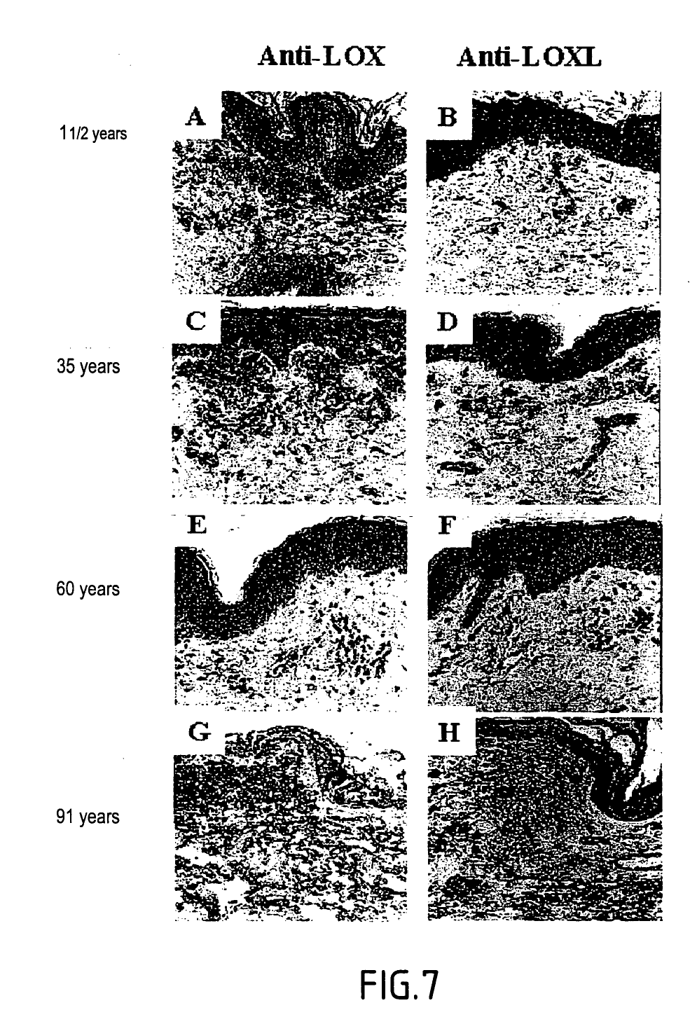

On FIG. 7: The immuno-detection of LOX and LOXL is represented in skins of the human abdomen taken at various ages.

The antibodies anti-LOX (A, C, E, G) and anti-LOXL (B, D, F, H) were used for detecting the expression of LOX and LOXL in samples of skin of the abdomen taken at 1.5 years old (A, B), 35 years old (C, D), 60 years old (E, F) and 91 years old (G, H) originating from the tissue bank of the Edouard Herriot Hospital. The tissues were fixed with Bouin's reagent, included in paraffin, and treated for the immuno-detection as described above.

During this study, a high expression was observed of LOX and LOXL in the epidermis of human skin, with an late extinction of the expression of these 2 enzymes (91 years old) (FIG. 7).

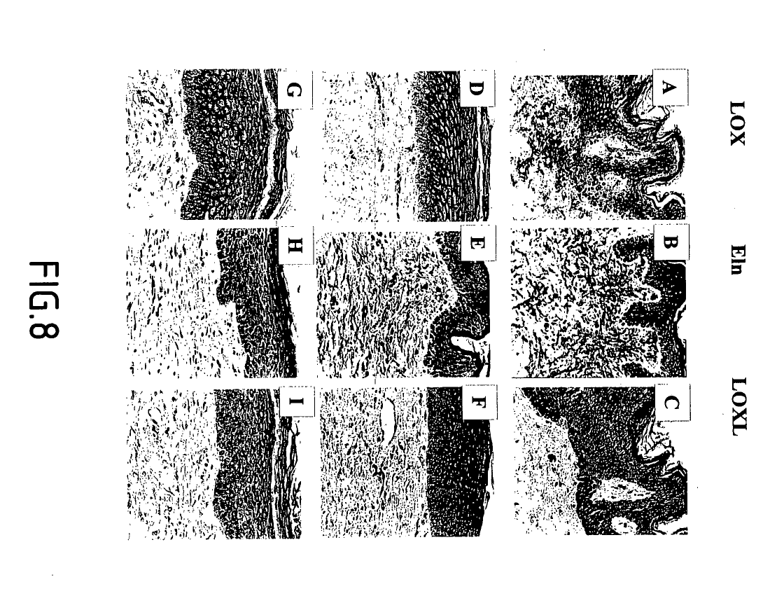

On FIG. 8: The immuno-detection of LOX and LOXL is represented in scar tissue skins at varying periods after the healing.

The antibodies anti-LOX (A, D, G), anti-elastin (B, E, H) and anti-LOXL (C, F, I) were used for detecting the expression of LOX of the elastin and of LOXL in samples of skin of the neck of a patient of 17 years old, around the scar (<<normal>> zone, A-C), 3 months (D-F) or 5 years old (G-H) after a healing. The tissues were fixed with Bouin's reagent, included in paraffin, and treated for the immuno-detection as described above.

In the scars, neither LOXL nor LOX were able to be observed in the scar tissue zones, 3 months after the scar and 5 years after the scar. It is to be noted that the elastin was immuno-detected at 3 months and disappeared at 5 years in these scars (FIG. 8).

This example of the invention demonstrates that there exists (i) an undeniable implication of LOX in the formation of tissues which are not in restructuring at various ages but (ii) a veritable deficit of expression of LOX, in the scars. This example demonstrates that LOX is present in the sites of formation of the elastic fibers but that it is absent in the restructured scar tissue zones which have lost the capacity to form functional elastic fibers.

### Example 7

### Demonstration of the Activation of LOX by the Introduction of Keratinocytes in a Reconstructed Skin Model

The activation of the gene encoding LOX is carried out by the in situ hybridization of the messenger RNA of LOX with double strand DNA probes which are labelled with digoxigenin, on sections included in paraffin.

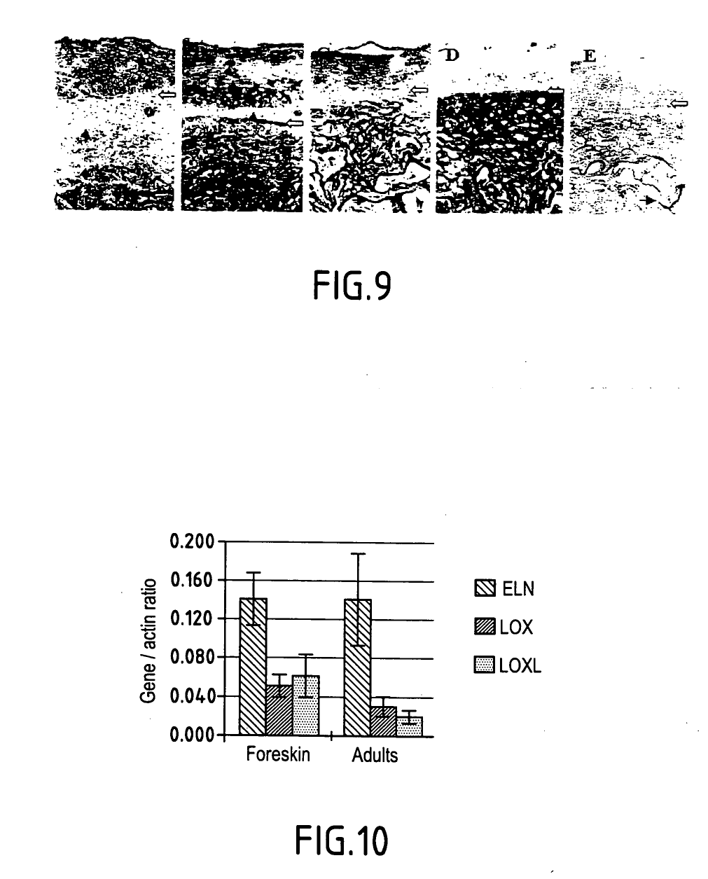

On FIG. 9, which represents skin model sections (Mimeskin®) at day 35:

(A) the expression of LOXL is positive in the deep dermis and throughout all the epidermis.

(B) The expression of LOX is observed throughout the whole dermis. In the epidermis, the cells of the basal layer do not express LOX, whereas the spinous and granular layers express it strongly.

(C) The expression of tropoelastin (TE) is found in association with the dermal fibroblasts and in the epidermis.

(D) The expression of the gene COL1A1 (collagen Iα1) is detected in the dermis but not in the epidermis.

(E) Control without probe.

The position of the DEJ is indicated by an open arrow, the position of the porous dermal substrate is indicated with arrows, and the positive cells are indicated with arrow heads.

The double strand DNA probes are produced by PCR. The following primers were used, respectively:

For the gene of the 1alpha1 collagen, sense 5′-GTGGAGAGTACTGGATTG-3′ (SEQ ID No14) and antisense 5′-TCGTGCAGCCATCGACAG-3′ (SEQ ID No15), for tropoelastin, sense 5′-GTATATACCCAGGTGGCGTG-3′ (SEQ ID NO10) and antisense 5′-CGMCTTTGCTGCTGCTTTAG-3′ (SEQ ID No11); for hLOX, sense 5′-GGTGGCCGACCCCTACTACATCC-3′ (SEQ ID No12) and antisense 5′-GCAAATCGCCTCTGGTAGCCATAGTC-3′ (SEQ ID No13); for hLOXL, sense 5′-GACATMCCGACGTGCAGCC-3′ (SEQ ID No8) and antisense 5′-ATCCACGTTCGCTCCCTGAG-3′ (SEQ ID No9).

The DNAs are amplified with Taq Polymerase (Promega, Charbonnieres, France) and Dig-11-dUTP (Roche Diagnostic, Meylan, France) as labeller nucleotide, and they are then purified after electrophoresis on agarose gel by using the QIAquick extraction kit (Qiagen, Courtaboeuf, France). The in situ hybridization was carried out on sections included in paraffin. The samples are ridded of paraffin and treated with proteinase K (Roche) at 2 μg/ml for 15 minutes at 20° C. The endogenous peroxidases are inhibited as indicated in the TSA+amplification kit (NEN, Boston, USA). A pre-hybridization is carried out for 2 hours at 37° C. in 20 mM phosphate buffer at pH 7.4, with 50% deionised formamide, 2×SSC (sodium salt citrate), 5 mM EDTA, 2.5× Denhardt's solution, 200 μg/ml denatured herring DNA, 1 mg/ml salmon sperm DNA, and 10 mg/ml tRNA. The hybridization is carried out for 16 hours at 37° C. in 20 mM phosphate buffer, with 50% deionised formamide, 2×SSC, 5 mM EDTA, 2.5× Denhardt's solution, 200 μg/ml denatured herring sperm DNA, 10% dextran sulphate, with or without the prior-denatured probe for 5 minutes in a boiling water bath. After the hybridization, the sections are washed at 20° C. (or 37° C. for the collagen) in 2×SSC/50% formamide, 1×SSC/50% formamide, 1×SSC, and 0.5×SSC. After dehydration, the hybrids labeled with the digoxigenin are detected with an anti-DIG antibody conjugated with horseradish peroxidase (Roche). The final detection of the complexes is carried out by using the TSA⁺ amplification kit (NEN). The positive signals correspond to the activity of the alkaline phosphatase linked to the amplification procedure of the TSA kit, after 2 hours of activity at ambient temperature, and defined by the precipitation of the tetrazolium salts formed (by using the Nitro Blue Tetrazolium/bromochlorylindolophosphate (NBT/BCIP) substrates).

The invention demonstrates that the LOXL and LOX genes can be activated by the addition of keratinocytes in a reconstructed skin model (Mimeskin®, Coletica, Lyons, France), as the tracking of the expression of the mRNAs by in situ hybridization demonstrates (FIG. 9). The LOX gene is also activated after the addition of the keratinocytes at the same time as the collagen 1 alpha1 gene (Collalphal).

### Example 8

### Demonstration of a Drop in the Level of the Expression of the LOX Gene in Aged Adult Fibroblasts

The inventors used five strains of fibroblasts from the foreskin (FF) (originating from young infants) and 6 strains of adult fibroblasts (AF, 3 of 20 years old on average, and 3 of 60 years old on average) originating from plastic surgery on the abdomen. The expression of the three genes of interest, as well as of actin, was analyzed by real time RT-PCR (quantitative reverse transcriptase polymerase chain reaction, FIG. 10). This technique enables precisely quantifying the expression of a gene in comparing it to that of the actin (considered as constant). The regulation of the level of this gene can therefore be quantified.

On FIG. 10: FIG. 10 represents the evolution of the expression of the genes of elastin, LOX and LOXL during ageing. These results were obtained by real time RT-PCR analysis of the level of expression of LOX, LOXL, and elastin in the fibroblasts of the foreskin and the adult. The results represent the average of the expression of each gene in five strains of FF and six strains of AF. The genes tested are hLOXL, hLOX and human elastin genes. The values of RT-PCR are compared to the amplification of the actin. It is noted that if the expression of the gene of the elastin does not seem to be disturbed during ageing, that of the LOX gene drops by 40% as from the age of 20. This piece of data is in agreement with the literature on elastin: if the elastic tissue deteriorates and is not replaced, it does not seem to be due to an inhibition of the activity of the elastin gene.

The total RNAs are purified with the <<SV 96Total RNA Isolation System >> kit (Promega, Charbonniéres, France). The purified RNAs are eluted in 100 μl of RNase-free water (Promega, Charbonniéres, France), determined and distributed in a plate (96 well, 10 μl total RNA at 5 ng/μl by PCR). The primers selected for the implementation of this work are the following and are the subject of Table I:

TABLE I

| | | | | Position on | Melting |
|---|---|---|---|---|---|
| | | Size | | the human | temperature |
| Gene | Name | (nucleotides) | Human sequence | gene | (MT) |
| ELN | 1 Ela | 20 | GTA TAT ACC CAG | Sense: +443 | 62° C. |
| | | | GTG GCG TG | | |
| | 2 Ela | 21 | CGA ACT TTG CTG | Antisense: +799 | 62° C. |
| | | | CTG CTT TAG | | |
| LOX | Ox 64 | 21 | ACG TAC GTG CAG | Sense: +676 | 60° C. |
| | | | AAG ATG TCC | | |
| | Ox 65 | 21 | GGC TGG GTA AGA | Antisense: +841 | 59° C. |
| | | | AAT CTG ATG | | |
| LOXL* | 30 L1 | 19 | GAC TTC GGC AAC | Sense: +1480 | 60° C. |
| | | | CTC AAC C | | |
| | 30 L2 | 20 | TGT TGC AGA AAC | Antisense: +1701 | 60° C. |
| | | | GTA GCG AC | | |
| ACTIN | Actin | 20 | GTG GGG CGC CCC | U sense | 72° C. |
| | | | AGG CAC CA | | |
| | Actin | 24 | CTC CTT AAT GTC | D antisense | 57° C. |
| | | | ACG CAC GAT TTC | | |

*With 30L1 being the sequence ID N° 28 and 30L2 the sequence ID N° 29.

The technique of real time RT-PCR is carried out with the <<Quanti Tect SYBR Green RT-PCR >> kit (Qiagen, France) on wells containing mRNA, in an OPTICON thermocycler, which carries out amplification cycles. The retrotranscription (RT) is performed for 30 minutes at 50° C., tracked for 15 minutes at 95° C. in order to inhibit the reverse transcriptase, to activate the polymerase and to denature the complementary DNA (cDNA) obtained. 50 chain polymerization cycles (PCR) are carried out (15 seconds at 94° C., 30 seconds at 60° C., 30 seconds at 72° C.). At every cycle end, the fluorescence, which is proportional to the number of fragments amplified, is read. The level of expression is defined by the ratio of expression of each gene with respect to actin.

The Examples above demonstrate that the synthesis of the products of the genes LOXL and LOX can be activated at gene level. The activation of the gene encoding LOX enables the formation of cross-linked collagen fibers. A screening of active principles enables leading to the identification of molecules which can induce, simultaneously or not, an increase of the expression of LOX and a decrease of the expression of the elastin, so as to stimulate the synthesis of cross-linked collagen while at the same time avoiding the formation of amorphous accumulations of elastin and with the view to re-inducing the expression of normal tissues in pathological tissues.

### Example 9

### Analysis of the Expression of the Messenger RNAs of LOX and/or of Elastin, for Example by Qualitative RT-PCR with or without the Placing in Contact of Active Principles the Activity of which is to be Tested

The actives were tested at 1% (v/v) on fibroblasts of normal human skin (originating from healthy <<aged >> adults). The culture was carried out, e.g. in a monolayer on 24-well culture plates, in a defined medium without serum (Fibroblast Basal Medium, Promocell, Germany). The cells were sown, e.g. at 40,000 per cm². At the confluence, the cells are placed in contact with the actives advantageously for 24 hours. In parallel, a non-treated control (medium alone) and three positive controls (TGF-β at 1 ng/ml, IL-1α at 50 pg/ml and Phytokine® (Coletica, France) at 2%(v/v)) are advantageously carried out, e.g. on the same culture plate.

The TGF-β at 1 ng/ml and the IL-1α at 50 pg/ml were tested beforehand and the stimulation of the synthesis of elastin mRNA induced by these two cytokines at these concentrations was verified by an analysis of the mRNAs, e.g. by quantitative RT-PCR (×10 for TGF-β and x6 for IL-1 alpha). After the time of placing the actives in contact with the cells, e.g. 24 hours, the media are removed and the cells are preserved e.g. by dry freezing at −80° C. after a rinsing in phosphate buffer pH 7.4. The total RNAs are extracted e.g. with the aid of an extraction kit of 96 wells on silica columns and were determined on a 96-well spectrophotometer at 260 nm (purity indicator: protein determination at 280 nm). The RNAs are diluted e.g. to 5 ng/μl. The qualitative RT-PCR in 1 step is carried out e.g. on 50 ng of initial RNA on a 96-well plate, on the genes of actin, elastin, and of LOX. The specific primers of each gene are used e.g. at 0.5 μM:

sense elastin gene: 1Ela 5′-GTA TAT ACC CAG GTG GCG TG-3′; (SEQ ID No26)

antisense elastin gene: 2Ela 5′-CGA ACT TTG CTG CTG CTT TAG-3′; (SEQ ID No27)

sense LOX gene: Ox64 5′-ACG TAC GTG CAG MG ATG TCC-3′; (SEQ ID NO24)

antisense LOX gene: Ox65 5′-GGC TGG GTA AGA MT CTG ATG-3′; (SEQ ID No25)

sense actin gene: Actin U 5′-GTGGGGCGCCCCAGGCACCA-3′; (SEQ ID No30)

antisense actin gene: Actin D 5′-CTCCTTAATGTCACGCACGATTTC-3′. (SEQ ID No31)

The amplification parameters were advantageously the following: 48° C., 30 min 94° C., 2 min; (94° C., 30 seconds; 60° C., 30 seconds; 68° C., 30 seconds) 28 cycles for actin, 32 cycles for LOX, or 34 cycles for elastin; 68° C., 10 min; 14° C., infinity. After amplification, the products are for example mixed at the rate of 3 μl of actin amplification products +5 μl of elastin gene amplification products +5 μl of LOX gene amplification products. A loading buffer is added (2 μl) and the total volume (20 μl) is deposited on a pre-poured agarose gel e.g. at 2%. The inventors visualized the level of expressions by means known to the person skilled in the art and e.g.: the bands of the samples were visualized under UV in a black chamber after migration (15 minutes) and were photographed digitally. The photographs of the gels were analyzed by image analysis and quantification of the intensity of the bands (Phoretix1D Quantifier, Non Linear Dynamics Ltd, USA). The levels of expression of the elastin genes and LOX genes were expressed in percentage variation with respect to those obtained for the negative control (without treatment).

Intergretations of the Results:

It is noted that the aged cells express quantities of mRNA encoding elastin which are roughly identical to those determined in the young cells, while they decrease very strongly in the case of the mRNA encoding LOX (−40%). It is therefore possible to reverse this decrease of the expression of LOX in the aged cells, and a screening of active principles in this sense was performed.

Screening of Active Principles

The amounts of cDNA of each test are compared to the amount of actin cDNA and then to the negative controls (without actives). A preliminary analysis enables considering the tests presenting an increase of LOX mRNA of about twice and an increase of elastin (Eln) mRNA of about 0.5, to be significant. Of more than 900 molecules or active extracts tested, 8 actives meet these criteria at the concentrations tested and under the defined conditions. These actives are the following and are the subject of Table II

TABLE II

| | Eln | LOX |
|---|---|---|
| Name | Control multiplied by: | Control multiplied by: |
| Polyphenols | 0.50 | 2.34 |
| Galega plant | 0.30 | 2.38 |
| Saturated fatty acid | 0.38 | 2.70 |
| Xylitol | 0.27 | 3.10 |
| Gum adragant | 0.33 | 4.10 |
| Cardamon | 0.36 | 2.64 |
| Barley | 0.31 | 4.65 |
| Oats | 0.38 | 9.69 |

The plant extracts were obtained by allowing the plants to soak at 2-5% (w/w) in a water/(alcohol, glycol or polyol) mixture (such as ethanol, glycerol, butylene glycol and other glycols, xylitol etc . . . ) 100/0 to 0/100. The extracts obtained were then filtered or distilled so as to recover the soluble fraction which is then filtered sterile. The chemical molecules originate from Sigma (Saint-Louis, USA) and are used diluted or dispersed at 1% in an alcohol or a glycol.

Conclusions: 8 actives from the bank of 960 actives are capable, under the conditions considered, of significantly activating the level of synthesis of mRNA of the genes encoding LOX and of decreasing the elastin in the fibroblasts of the abdomen of the <<aged >> adult.

### Example 10

### Study of Effectiveness of a Cosmetic or Dermopharmaceutical Active by e.g. Real Time RT-PCR

The actives selected after the first step of screening were tested at various concentrations of between 0.001% and 5%, (v/v) on fibroblasts of normal (adult) human skin. The culture was carried out e.g. in monolayer in 24-well plates, in a defined medium without serum (Fibroblast Basal Medium, Promocell, Germany). The cells are sown e.g. at 40,000 per cm². After the time of placing the actives in contact with the cells (24 hours), the media are removed and the cells were preserved e.g. by dry freezing at −80° C. after a rinsing with phosphate buffer at pH 7.4. At the end of experimentation, the content of mRNA of elastin, of LOX and of actin is evaluated by an mRNA analysis technique, e.g. by real time RT-PCR. For this, the couples of primers enabling the amplification of specific fragments of these genes are those described above (Example 9).

After extraction e.g. with the aid of an extraction kit in 96-wells on silica columns and determination on a 96-well spectrophotometer at 260 nm, the RNAs are diluted e.g. to 5 ng/μl. The RT-PCR reactions (Reverse Polymerase Transcription Chain Reactions) were carried out by quantitative real time RT-PCR with the aid of the “Opticon” system (MJ Research, USA). Advantageously, the reaction mixture (50 μl) introduced into the well was the following, for each sample:

10 μl of RNA at a concentration of 5 ng/pl,

The specific primers of the various labels sought after,

Reaction mixture (Qiagen −25 μl 2×QuantiTect SYBR Green RT-PCR master mix containing 5 mM MgCl2+0.5 μl QuantiTect RT mix), the label SYBR Green I inserting in the DNA double strands during the elongation step.

The RT-PCR conditions were advantageously the following:

Reverse Transcription: 30 minutes at 50° C., then 15 minutes at 95° C., PCR reactions: [15 seconds at 94° C., 30 seconds at 60° C. and 30 seconds at 72° C.], 50 cycles.

The absence of contamination and the purity of the amplified products were verified e.g. via the fusion curves of the amplified PCR products. The products presenting a double peak or an abnormal fusion temperature, were eliminated.

Analysis and Method of Calculation

The incorporation of fluorescence in the amplified DNA was evaluated continuously during the PCR cycles. This system enabled obtaining curves of fluorescence measurement as a function of the number of PCR cycles and thus enabled evaluating a relative amount of amplified DNA.

In order to take account of the cell population present, all the results were compared to the <<actin >> signal, which was used as housekeeping gene. According to the experimentation, the threshold of measurement of the C M (=Cycle Threshold) was fixed for T between 0.05 and 0.01, and then an arbitrary measurement unit is calculated for each gene according to the formula

Sgene <<x>>=10⁷×(1/2)^{C(T)gene<<x>>}

C (T)gene <<x>>signifying the number of cycles necessary to attain the fluorescence threshold of 0.01-0.05 of the gene <<x>>.

The values of the genes of interest were compared to the <<actin >> signal by calculation of the ratio:

R=Sgene <<x>>/Sactin.

These ratios were compared between the treated and non-treated samples, “x” being the LOX gene, or the elastin gene.

Results: amongst the actives-selected, the results obtained, as an example for two of them, are presented in Table III

TABLE III

| | LOX | Eln |
|---|---|---|
| Name | Control multiplied by: | Control multiplied by: |
| Cardamon at 0.001% | 1.38* | 0.67* |
| Cardamon at 0.01% | 1.48* | 0.69* |
| Cardamon at 0.1% | 1.63* | 0.64* |
| Cardamon at 1% | 1.64* | 0.26* |
| Cardamon at 5% | 2.06* | 0.41* |
| Oats at 0.001% | 1.41 | 1.17 |
| Oats at 0.01% | 2.06 | 0.76 |
| Oats at 0.1% | 1.84* | 0.97 |
| Oats at 1% | 2.25* | 0.81 |
| Oats at 5% | 1.40 | 0.39* |

*statistically significant results p < 0.05 (One Way test Anova/Bonferroni)

Conclusions: The extract of cardamon significantly STIMULATES LOX at all the concentrations and significantly INHIBITS elastin (Eln) at all the concentrations. The extract of oats significantly STIMULATES LOX at 0.1% and 1% and significantly INHIBITS elastin (Eln) at 5%.

### Example 11

### Analysis of the Expression of the Messenger RNAs of LOX and/or of Elastin, e.g. by Qualitative RT-PCR with or without Placing in Contact of Active Principles the Activity of which is to be Tested on Cells Originating from Scar Tissue Zone

The actives were tested at 1% (v/v) on human scar tissue skin fibroblasts (surgical recovery of hypertropic scar after caesarean) in premature passage (lower than 5 passages). The culture was carried out for example in a monolayer on 24-well culture plates, in a defined medium without serum (Fibroblast Basal Medium, Promocell, Germany). The cells were sown, e.g. at 40,000 per cm². At the confluence, the cells are placed in contact with the actives advantageously for 24 hours. In parallel, a non-treated control (medium alone) and three positive controls (TGF-β at 1 ng/ml, IL-1α at 50 pg/ml and Phytokines (Coletica, France) at 2%(v/v)) are advantageously carried out, e.g. on the same culture plate. The TGF-β at 1 ng/ml and the IL-1α at 50 pg/ml were tested beforehand and the stimulation of the synthesis of elastin mRNA induced by these two cytokines at these concentrations was verified by an analysis of the mRNAs, e.g. by quantitative RT-PCR (x10 for TGF-β and x6 for IL-1 alpha). After the time of placing the active principles in contact with the cells, e.g. 24 hours, the media are removed and the cells are preserved e.g. by dry freezing at −80° C. after a rinsing in phosphate buffer pH 7.4. The total RNAs are extracted e.g. with the aid of an extraction kit of 96 wells on silica columns and were determined on a 96-well spectrophotometer at 260 nm (purity indicator: protein determination at 280 nm). The RNAs are diluted e.g. to 5 ng/μl. The qualitative RT-PCR in 1 step is carried out e.g. on 50 ng of initial RNA on a 96-well plate, on the genes of actin, collagen I alpha1 and LOX. The specific primers of each gene were used e.g. at 0.5 μM

| | |
|---|---|
| COLL1 sense | 5′-CAG AGG GAA GCC GCA AGA-3′ |
| COLL1 | 5′-CTG GCC GCC ATA CTC GAA C-3′ |
| antisense | |
| LOX sense | 5′-ACG TAC GTG CAG AAG ATG TCC-3′ |
| LOX antisense | 5′-GGC TGG GTA AGA AAT CTG ATG-3′ |
| Actin sense | 5′-GTG GGG CGC CCC AGG CAC CA-3′ |
| Actin | 5′-CTC CTT AAT GTC ACG CAC GAT TTC-3′ |
| antisense | |

The amplification parameters were advantageously the following: 50° C., 30 min 94° C., 2 min; (94° C., 30 seconds; 60° C., 30 seconds; 68° C., 30 seconds) 28 cycles for actin, 26 cycles for COLL1, 32 cycles for LOX; 72° C., 10 min; 14° C., infinity. After amplification, the products are for example mixed at the rate of 3 μl of actin amplification products+5 μl of collagen I gene amplification products +5 μl of LOX gene amplification products. A loading buffer is added (2 μl) and the total volume (20 μl) is deposited on a pre-poured agarose gel e.g. at 2%. The inventors visualized the level of expressions by means known to the person skilled in the art and e.g.: the bands of the samples were visualized under UV in a black chamber after migration (15 minutes) and were photographed digitally. The photographs of the gels were analyzed by image analysis and quantification of the intensity of the bands. The level of expression of the collagen I genes and LOX genes were expressed in percentage variation with respect to those obtained for the negative control (without treatment) and were compared to the results obtained for the positive controls.

The actives selected present an increase of LOX mRNA of about twice and a normal or reduced expression of the COL1 mRNA. Of over 900 active molecules or extracts tested, an extract of oats particularly meets positively these criteria.

For Examples 12 to 16: the person skilled in the art will know how to draw the adequate teaching from these Examples in order to make the various compositions (formulations) described.

### Example 12

### Use of the Products of the Invention in Cosmetic or Pharmaceutical Formulations of Oil-in-Water Emulsion Type

Formulation 12a:

| A | Water | qsp 100 |
|---|---|---|
| | Butylene Glycol | 2 |
| | Glycerine | 3 |
| | Sodium Dihydroxycetyl | 2 |
| | Phosphate, | |
| | Isopropyl Hydroxycetyl Ether | |
| B | Glycol Stearate SE | 14 |
| | Triisononaoin | 5 |
| | Octyl Cocoate | 6 |
| C | Butylene Glycol, | 2 |
| | Methylparaben, | |
| | Ethylparaben, Propylparaben, | |
| | pH adjusted to 5.5 | |
| D | Products of the invention | 0.01-10% |

Formulation 12b:

| | | |
|---|---|---|
| A | Water | qsp 100 |
| | Butylene Glycol | 2 |
| | Glycerine | 3 |
| | Polyacrylamide, Isoparaffin, | 2.8 |
| | Laureth-7 | |
| B | Butylene Glycol, | 2 |
| | Methylparaben, | |
| | Ethylparaben, Propylparaben; | 2 |
| | Phenoxyethanol, | |
| | Methylparaben, | |
| | Propylparaben, Butylparaben, | |
| | Ethylparaben | 0.5 |
| | Butylene Glycol | |
| D | Products of the invention | 0.01-10% |

Formulation 12c:

| | | |
|---|---|---|
| A | Carbomer | 0.50 |
| | Propylene Glycol | 3 |
| | Glycerol | 5 |
| | water | qsp 100 |
| B | Octyl Cocoate | 5 |
| | Bisabolol | 0.30 |
| | Dimethicone | 0.30 |
| C | Sodium Hydroxide | 1.60 |
| D | Phenoxyethanol, | 0.50 |
| | Methylparaben, | |
| | Propylparaben, Butylparaben, | |
| | Ethylparaben | |
| E | Perfume | 0.30 |
| F | Products of the invention | 0.01-10% |

### Example 13

### Use of the Products of the Invention in a Water-in-Oil Type Formulation

| | | |
|---|---|---|
| A | PEG 30 - | 3 |
| | dipolyhydroxystearate | |
| | Capric Triglycerides | 3 |
| | Cetearyl Octanoate | 4 |
| | Dibutyl Adipate | 3 |
| | Grape Seed Oil | 1.5 |
| | Jojoba Oil | 1.5 |
| | Phenoxyethanol, | 0.5 |
| | Methylparaben, | |
| | Propylparaben, Butylparaben, | |
| | Ethylparaben | |
| B | Glycerine | 3 |
| | Butylene Glycol | 3 |
| | Magnesium Sulphate | 0.5 |
| | EDTA | 0.05 |
| | Water | qsp 100 |
| C | Cyclomethicone | 1 |
| | Dimethicone | 1 |
| D | Perfume | 0.3 |
| E | Products of the invention | 0.01-10% |

### Example 14

### Use of the Products of the Invention in a Formulation of Shampoo or Shower Gel Type

| | | |
|---|---|---|
| A | Xantham Gum | 0.8 |
| | Water | qsp 100 |
| B | Butylene Glycol, | 0.5 |
| | Methylparaben, | |
| | Ethylparaben, Propylparaben | |
| | Phenoxyethanol, | 0.5 |
| | Methylparaben, | |
| | Propylparaben, Butylparaben, | |
| | Ethylparaben | |
| C | Citric acid | 0.8 |
| D | Sodium Laureth Sulphate | 40.0 |
| E | Product of the invention | 0.01-10% |

### Example 15

### Use of the Products of the Invention in a Formulation of Lipstick and Other Anhydrous Product Type

| | | |
|---|---|---|
| A | Mineral Wax | 17.0 |
| | Isostearyl Isostearate | 31.5 |
| | Propylene Glycol Dipelargonate | 2.6 |
| | Propylene Glycol Isostearate | 1.7 |
| | PEG 8 Beeswax | 3.0 |
| | Hydrogenated Palm Kernel Oil | 3.4 |
| | Glycerides, | |
| | Hydrogenated Palm Glycerides | |
| | Lanolin Oil | 3.4 |
| | Sesame Oil | 1.7 |
| | Cetyl Lactate | 1.7 |
| | Mineral Oil, Lanolin Alcohol | 3.0 |
| B | Castor Oil | qsp 100 |
| | Titanium Dioxide | 3.9 |
| | CI 15850:1 | 0.616 |
| | CI 45410:1 | 0.256 |
| | CI 19140:1 | 0.048 |
| | CI 77491 | 2.048 |
| C | Products of the invention | 0.01-5% |

### Example 16

### Use of the Products of the Invention in a Formulation of Aqueous Gels (Eyeliners, Slimmers, etc.)

| A | Water | qsp 100 |
|---|---|---|
| | Carbomer | 0.5 |
| | Butylene Glycol | 15 |
| | Phenoxyethanol, Methylparaben, | 0.5 |
| | Propylparaben, Butylparaben, | |
| | Ethylparaben | |
| B | Products of the invention | 0.01-10% |

### Example 17

### Preparation of Pharmaceutical Formulations Containing

Formulation 17A: preparation of tablets

| A | Excipients | In g, per tablet |
|---|---|---|
| | Lactose | 0.359 |
| | Sucrose | 0.240 |
| B | Extract of LOX* | 0.001-0.1 |

*The extract of LOX is obtained for example according to the method of extraction described in Example 2, followed by a drying step.

Formulation 17b: preparation of an ointment

| A | Excipients | |
|---|---|---|
| | Low density polyethylene | 5.5 |
| | Liquid paraffin | qsp 100 |
| B | Extract of LOX* | 0.001-0.1 |

*The extract of LOX is obtained for example according to the method of extraction described in Example 2, optionally followed by a drying step.

Formulation 17c: preparation of an injectable formula

| A | Excipient | | |
|---|---|---|---|
| | Saline isotonic solution | 5 | ml |
| B | Extract of LOX* | 0.001-0.1 | g |

*The extract of LOX is obtained for example according to the method of extraction described in Example 2, followed by a drying step.

Phase A and Phase B are packaged in separate ampoules and are mixed before use.

### Example 18

### Evaluation of the Cosmetic Acceptance of a Preparation Containing the Subject of the Invention

Toxicology tests were carried out on the compound obtained according to Examples 9 to 11 incorporated at 10% in a 0.5% xanthan gum, by an ocular evaluation in the rabbit, by the study of the absence of abnormal toxicity by single oral administration in the rat and by the study of the sensitizing power in the guinea pig.

Evaluation of the Primary Irritation of the Skin in the Rabbit:

The preparations described above were applied without dilution at the dose of 0.5 ml on the skin of 3 rabbits according to the method recommended by the OECD in relation to the study of <<the acute irritant/corrosive effect on the skin >>.

The products are classed according to the criteria defined in the Decision of Jan. 2, 1982 published in the Official Journal of the French Republic (the “JORF”) of 21/02/82.

The results of these tests have enabled concluding that the preparation containing the compound obtained according to Example 11 was classed as non-irritant for the skin.

Evaluation of the Ocular Irritation in the Rabbit:

The preparations described above were instilled pure and in one batch at the rate of 0.1 ml in the eye of three rabbits according to the method recommended by the directive of the OECD NO. 405 of Feb. 24, 1987, in relation to the study of “the acute irritant/corrosive effect on the eyes”.

The results of this test enable concluding that the preparations can be considered as non-irritant for the eyes, in the sense of the Directive 91/326 EEC, used pure or without dilution.

Test on the Absence of Abnormal Toxicity by Single Oral Administration in the Rat:

The preparations described were administered in one batch orally at the dose of 5 g/Kg of body weight, to 5 male rats and 5 female rats of a protocol inspired from the Directive of the OECD No. 401 of Feb. 24, 1987 and adapted to cosmetic products.

The LD0 and LD50 are found to be greater than 5,000 mg/Kg. The preparations tested are therefore not classed amongst the preparations which are dangerous by ingestion.

Evaluation of the Skin Sensitisation Potential in the Guinea Pig:

The preparations described are subjected to the maximization test described by Magnusson and Kligmann, a protocol which is in agreement with the directive line No. 406 of the OECD.

The preparations are classed as non-sensitizing by contact with the skin. Precisions on the sequences which are described

Sequence ID No1: is the peptide sequence of the human protein LOX.

Sequence ID No2: is the sequence of nucleotides of the cDNA encoding the human protein LOX described in sequence ID No1.

Sequence ID No3: is the peptide sequence of the human protein tropoelastin.

Sequence ID No4: is the sequence-of nucleotides of the cDNA encoding the promoter (base pairs from −2171 up from ATG) of the human gene encoding the human elastin protein.

Sequence ID No5: is the sequence of nucleotides of the cDNA encoding the human protein tropoelastin described in sequence ID No3.

Sequence ID No6: is the peptide sequence of the human protein LOXL.

Sequence ID No7: is the sequence of nucleotides of the cDNA encoding the human protein LOXL described in sequence ID NO6.

For the double strand DNA probes:

Sequence ID No8: is a sense primer of the DNA encoding the human protein LOXL described in sequence ID NO6.

Sequence ID No9: is an antisense primer of the DNA encoding the human protein LOXL described in sequence ID NO6.

Sequence ID No10: is a sense primer of the DNA encoding the human protein tropoelastin described in sequence ID NO3.

Sequence ID No11: is an antisense primer of the DNA encoding the human protein tropoelastin described in sequence ID No3

Sequence ID No12: is a sense primer of the DNA encoding the human protein LOX described in sequence ID NO1.

Sequence ID No13: is an antisense primer of the DNA encoding the human protein LOX described in sequence ID NO1.

Sequence ID No14: is a sense primer of the DNA encoding the human protein collagen I α1L.

Sequence ID No15: is an antisense primer of the DNA encoding the human protein collagen I α1L.

For the GST fusion genes

Sequence ID No16: is a sense primer of the DNA of the fusion gene GST S355-D415.

Sequence ID No17: is an antisense primer of the DNA of the fusion gene GST S355-D415.

Sequence ID No18: is a sense primer of the DNA of the fusion gene GST G128-L212.

Sequence ID No19: is an antisense primer of the DNA of the fusion gene GST G128-L212.

Sequence ID No20: is a sense primer of the DNA of the fusion gene GST V228-S279.

Sequence ID No21: is an antisense primer of the DNA of the fusion gene GST V228-5279.

Sequence ID No22: is a sense primer of the DNA of the fusion gene GST D306-N373.

Sequence ID No23: is an antisense primer of the DNA of the fusion gene GST D306-N373.

For the PCR primers:

Sequence ID No24: is a sense primer for the RT-PCR of the mRNA encoding the human protein LOX described in sequence ID No1.

Sequence ID No25: is an antisense primer for the RT-PCR of the mRNA encoding the human protein LOX described in sequence ID No1.

Sequence ID No26: is a sense primer for the RT-PCR of the mRNA encoding the human protein tropoelastin described in sequence ID No3.

Sequence ID No27: is an antisense primer for the RT-PCR of the mRNA encoding the human protein tropoelastin described in sequence ID No3

Sequence ID No28: is a sense primer for the RT-PCR of the mRNA encoding the human protein LOXL described in sequence ID No6.

Sequence ID No29: is an antisense primer of the sequence of the mRNA encoding the human protein LOXL described in sequence ID NO6.

Sequence ID No30: is a sense primer for the RT-PCR of the mRNA encoding the human protein actin.

Sequence ID No31: is an antisense primer for the RT-PCR of the mRNA encoding the human protein actin.

1. A composition for regulating elastogenesis in cases of abnormal or pathological elastogenesis comprising the lysyl oxidase isoform LOX having the Sequence ID No1, or of an homologous or essentially homologous form thereof.

2. A composition for regulating elastogenesis in cases of abnormal or pathological elastogenesis comprising a first substance stimulating LOX or of an homologous form thereof, combined with a second substance promoting the reduction or the inhibition of elastin synthesis.

3. A composition of claim 1, wherein the abnormal or the pathological elastogenesis is encountered in cases of fibrosis, of solar elastosis, of stretch marks, of dystrophic scars, of eczemas, of a stromal reaction to cancers which are associated with a disorganized or non-functional elastic tissue.

4. The composition of claim 2, wherein the first substance and the second substance are identical.

5. The composition of claim 2, wherein said second substance promotes the reduction or the inhibition of the synthesis of tropoelastin having the sequence ID No3 or of an homologous or essentially homologous form thereof, which is the precursor protein of elastin.

6. The composition of claim 2, wherein said second substance comprises a region which fixes to at least one part of the nucleotide sequence of the human elastin gene promoter (Pr) (SEQ ID No4) or of an homologous or essentially homologous form thereof so as to reduce or to inhibit the synthesis of tropoelastin.

7. The composition of claim 2, wherein said second substance modulates the expression of a protein which fixes to at least one part of nucleotides sequence of the human elastin gene promoter (Pr) (SEQ ID No4) or of an homologous or essentially homologous form thereof, so as to reduce or to inhibit the synthesis of tropoelastin.

8. The composition of claim 1, wherein said active substance is selected from the group consisting of plant or synthetic polyphenols, an extract of galega, an extract of cardamon, a saturated fatty acid, a polyol, xylitol, gum adragant, an extract of cereals, barley and oats.

9. The composition of claim 2, wherein said active substance is selected from the group consisting of plant or synthetic polyphenols, an extract of galega, an extract of cardamon, a saturated fatty acid, a polyol, xylitol, gum adragant, an extract of cereals, barley and oats.

10. A cosmetic composition comprising an active substance in claim 1, in admixture with a cosmetically acceptable excipient.

11. A cosmetic composition comprising an active substance in claim 2, in admixture with a cosmetically acceptable excipient.

12. A neutraceutical composition, comprising an active substance as defined in claims 1, in admixture with an excipient acceptable for food.

13. A neutraceutical composition, comprising an active substance as defined in claims 2, in admixture with an excipient acceptable for food.

14. A pharmaceutical composition, comprising an active substance as defined in claim 1, in admixture with a pharmaceutically acceptable excipient.

15. A pharmaceutical composition, comprising an active substance as defined in claim 2, in admixture with a pharmaceutically acceptable excipient.

16. A screening method for screening a substance stimulating lysyl oxidase isoform LOX or of an homologous or essentially homologous form thereof, wherein said method comprises:
placing a potentially active substance in contact with at least one type of cells capable of expressing LOX or of an homologous or essentially homologous form thereof, and
analyzing the expression of LOX or of an homologous or essentially homologous form thereof, with the aim of identifying whether said potentially active substance stimulates the expression of LOX or of an homologous or essentially homologous form thereof.

17. The screening method of claim 16, wherein said method comprises placing said potentially active substance in contact with at least one type of cells capable of expressing the protein elastin, and comprises analyzing the expression of the protein elastin, with the aim of identifying whether said potentially active substance promotes the reduction or the inhibition of the synthesis of elastin.

18. The screening method of claim 16, wherein it is sought whether said potentially active substance stimulates the expression of a sequence selected from the group consisting of:
nucleotide sequence encoding the protein LOX; and
peptide sequence present within the structure of the protein LOX; and
said potentially active substance inhibits the expression of a sequence selected from the group consisting of
nucleotide sequence encoding the protein elastin, and
a peptides sequence which is present within the structure of the protein elastin.

19. The screening method of claim 16, wherein said analysis of the expression of LOX is carried out by qualitative or quantitative analysis of the expression of nucleotide sequence encoding LOX.

20. The screening method of claim 19, wherein said analysis of the expression of LOX uses of a reverse transcription polymerase chain reaction (RT-PCR) comprising the use of primers hybridizing with at least one part of the nucleotide sequence of the complementary DNA encoding LOX (SEQ ID No2), in order to amplify at least one part of the nucleotide sequence encoding LOX.

21. The screening method of claim 16, comprising a step of location of the expression of LOX which is carried out on a reconstructed skin model or on a model based on biopsies, by a method selected from the group consisting of in situ hybridization of at least one part of a nucleotide sequence encoding LOX, and immuno-detection of at least one part of a peptide sequence encoding LOX.

22. The screening method claim 16, comprising a step of location of the expression of LOX and of elastin, which is carried out on a reconstructed skin model or on a model based on biopsies, by a method selected from the group consisting of in situ hybridization of at least one part of a nucleotide sequence encoding LOX and of at least on part of nucleotide sequence encoding tropoelastin, immuno-dectection of at least one part of a peptide sequence encoding LOX and of at least one part of a peptide sequence encoding elastin.

23. The screening method of claim 21, wherein the step immuno-dectection or in situ hybridization enables performing the traceability of LOX in a tissue selected from the group consisting of epithelial tissues and connective tissues.

24. The screening method of claim 16, comprising the comparison of the expression of LOX with the expression of LOX expressed in a control which does not comprise said potentially active substance

25. The screening method of claim 16, wherein said cells comprise cells selected from the group consisting of fibroblasts, fibroblasts originating from normal human skin, fibroblasts originating from the foreskin, fibroblasts originating from a skin of an adult subject, epithelial cells, keratinocytes, epithelial cells originating from a normal human skin, epithelial cells originating from the foreskin, and epithelial cells originating from a skin of an adult subject.

26. The screening method of claim 16, wherein said cells originate from at least one skin having a particular location, said location being the face, the abdomen, the breasts, and being able to be characterized as being <<photo-aged >> or as being <<punctually exposed >> to sun's radiation or not.

27. The method of claims 16, comprising the use of a model selected from the group consisting of a reconstructed skin model, a model based on biopsies, a dermis model comprising fibroblasts, and an epidermis model comprising keratinocytes, for placing in contact said potentially active substance with said cells.

28. A method of locating the expression of LOX or of an homologous or essentially homologous form thereof in tissues with the aim of performing the traceability of neo-collagenogenesis or of neo-elastogenesis, wherein said method comprises a step of immuno-detection of the protein LOX.

29. A method of locating the expression of LOX or of an homologous or essentially homologous form thereof in tissues with the aim of performing the traceability of neo-collagenogenesis or of neo-elastogenesis, wherein said method comprises in situ hybridizing the gene encoding LOX.

30. A method of cosmetic care comprising delivering to a person the composition of claim 10.

31. A method of cosmetic care comprising delivering to a person the composition of claim 11.

32. The method of cosmetic care of claim 30, wherein said cosmetic care is selected from the group consisting of combating solar elastosis, combating stretch marks, combating dystrophic scars.

33. The method of cosmetic care of claim 31, wherein said cosmetic care is selected from the group consisting of combating solar elastosis, combating stretch marks, combating dystrophic scars.

34. A method of treatment of a disorder associated with the enzymatic activity of the isoform of the protein lysyl oxidase LOX or of an homologous or essentially homologous form thereof, comprising administering to a subject in need thereof a therapeutically effective amount of a composition comprising a substance stimulating the protein lysyl oxidase LOX or of an homologous or essentially homologous form thereof, said stimulation being selected from the group consisting of stimulating the enzymatic activity of the protein lysyl oxidase LOX or of an homologous or essentially homologous form thereof, and stimulating the expression of the protein lysyl oxidase LOX or of an homologous or essentially homologous form thereof.

35. The method of claim 34, wherein said treatment is selected from the group consisting of regulating elastogenesis, regulating pathological elastogenesis, regulating disorganized elastogenesis, regulating non-functional elastogenesis, in case of a disorder selected from the group consisting of fibrosis, solar elastosis, stretch marks, dystrophic scars, eczema, and a stromal reaction to cancers which are associated with a disorganized or non-functional elastic tissue.

36. A composition of claim 2, wherein the abnormal or the pathological elastogenesis is encountered in cases of fibrosis, of solar elastosis, of stretch marks, of dystrophic scars, of eczemas, of a stromal reaction to cancers which are associated with a disorganized or non-functional elastic tissue.


SEQUENCE LISTING (31 sequences)

SEQ ID NO: 1 - protein, 417 aa, Homo sapiens [sequence omitted]

SEQ ID NO: 2 - DNA, 1254 bp, Homo sapiens
atgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacgtggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattag

SEQ ID NO: 3 - protein, 788 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGAAGAGVLGGLVPGPQAAVPGVPGTGGVPGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 4 - DNA, 2263 bp, Homo sapiens
ctcgagaagagagggctccagctccccacagtagcccctgccttcctccttcccaggcaggcggaggcacgcagatataccattgacttcccctcccctgcagcaggcacatcctgggcatcgagcttcagaccctgcccctgagcagcccctaaccccaccaacaaagggtggcttgggggggctttcaccccagcataatctccatcagctaccctcaaagcacccccaaataaacacacaccgtaagtaagagctgtacactggctgtgtgcgtacatcttcaagacaattctcccagcatgcccctaccttccaaaattccagagctgctccctccaaagacccagggaaaaggaagggtttgtccagggtcctggggtggccccgtatagaccaaagcctgataactgtcctagaagcagagtacttgcagagcgagtgacggcaactgtggtattgacaccagtcctagcaccagctgaacacagagcatttttgatctagcagaaatacaagaccacgttgtatttgtctttgcaataatctcttagctaggaatactgatcacctgtagacagataaggaaactgatgctctgtggagaggttttcctaccagaaaggctagagccagaaatttacttctaggtccaccaatacctgcctttgaccaatgcctgcatttgacctttccacgctgagccacccctgctggcactccagactgccacagtgctcctgcctccacaaggggtctttaactcatccctcggagccatcgtggtgcagggaaaagcccacagggcgtgtggcttccatgctgttccctgactggctgtgacctaggacaaggaacaagtttccctctcctattctctaggtctcacatttcttctcctctagcagtagtgggaagtgaggggtgggggacacgaccctcccctgttccatcccacactccaacccccaaaatcccccagggtccccgtccagctcagtcctgggggcagaaatgcagagttctccaggaacgtggtcccagctgtttcagtgcaggccgccccctcctggccaccagcggaatgtcagccttcccagaggggccgggagaacagcagtcgagaagctcccagactggtgtgggcgctagctgtgctcagcgtggggatgggaggtgacccagtgataatgggaagctgggctgcctgtcagtctgtggggggctcccacctccctgttcccccacagggcacctggggatccagcctgatttttaccagacctgcggcctgcatggggctgggtatagggctgtgaccttgacccatgcagaatagaaccctgtgtgtcgggatcctccatgtgctccagatgcccctggggacagcaccaacatggccttaactcccaagccattcccctgcctctaaccccctggcatctgcaggcatccaccccagacccacccaacacctcctccccagcttcaggcgctaggcagagaccttggcccctgcagaatgcagccctgtccagggtcccctaccttccccccagatccctcccagagcaataccaacccgggcctaccttccaggccattcaacctgcagccccccggcctctgtagacatcgcaccccccaaacccccagacctgcccaatgcctcccctccccagctttgggcagaacctgtctctagccagacctgggggtgttggggagtctggagggccggggtgggggctgaggcgcgggacagctggcccgtatcctcacactgggcccagccggaggggcgggggcctggccactcgggccttggctggggctgggatttttggcctggccgccaggccctcccttctgcttcctctcccgagggctgtcctggcagaggcccccctcgctctttctggcgggaacagggccagcagcgaaagaacagtcgcagagggaaagcgggaaagagatgggggaaagtgtgtgtgtgtgagtgtgtgcttgtgtgcatgtgtgtgcgtgtgttgtgtcaagaaaaaagctcgcagtccagcagcccgggcctgggaggcttgtgagccgggcctttcgtaattgtcccctccccgcggccccctcccccaggcctcccccctctcccgccctcccgcccgccctctctccctccctctttccctcacagccgacgaggcaacaattaggctttggggataaaacgaggtgcggagagcgggctggggcatttctccccgagatg

SEQ ID NO: 5 - DNA, 2367 bp, Homo sapiens
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggtgctgcaggagcaggagtgctgggtgggctagtgccaggtccccaggcggcagtcccaggtgtgccgggcacgggaggagtgccaggagtggggaccccagcagctgcagctgctaaagcagccgccaaagccgcccagtttgggttagttcctggtgtcggggtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccgatgagggagttaggcggagcctgtcccctgagctcagggaaggagatccctcctcctctcagcacctccccagcaccccctcatcacccagggtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 6 - protein, 574 aa, Homo sapiens
MALARGSRQLGALVWGACLCVLVHGQQAQPGQGSDPARWRQLIQWENNGQVYSLLNSGSEYVPAGPQRSESSSRVLLAGAPQAQQRRSHGSPRRRQAPSLPLPGRVGSDTVRGQARHPFGFGQVPDNWREVAVGDSTGMALARTSVSQQRHGGSASSVSASAFASTYRQQPSYPQQFPYPQAPFVSQYENYDPASRTYDQGFVYYRPAGGGVGAGAAAVASAGVIYPYQPRARYEEYGGGEELPEYPPQGFYPAPERPYVPPPPPPPDGLDRRYSHSLYSEGTPGFEQAYPDPGPEAAQAHGGDPRLGWYPPYANPPPEAYGPPRALEPPYLPVRSSDTPPPGGERNGAQQGRLSVGSVYRPNQNGRGLPDLVPDPNYVQASTYVQRAHLYSLRCAAEEKCLASTAYAPEATDYDVRVLLRFPQRVKNQGTADFLPNRPRHTWEWHSCHQHYHSMDEFSHYDLLDAATGKKVAEGHKASFCLEDSTCDFGNLKRYACTSHTQGLSPGCYDTYNADIDCQWIDITDVQPGNYILKVHVNPKYIVLESDFTNNVVRCNIHYTGRYVSATNCKIVQS

SEQ ID NO: 7 - DNA, 1725 bp, Homo sapiens
atggctctggcccgaggcagccggcagctgggggccctggtgtggggcgcctgcctgtgcgtgctggtgcacgggcagcaggcgcagcccgggcagggctcggaccccgcccgctggcggcagctgatccagtgggagaacaacgggcaggtgtacagcttgctcaactcgggctcagagtacgtgccggccggacctcagcgctccgagagtagctcccgggtgctgctggccggcgcgccccaggcccagcagcggcgcagccacgggagcccccggcgtcggcaggcgccgtccctgcccctgccggggcgcgtgggctcggacaccgtgcgcggccaggcgcggcacccattcggctttggccaggtgcccgacaactggcgcgaggtggccgtcggggacagcacgggcatggccctggcccgcacctccgtctcccagcaacggcacgggggctccgcctcctcggtctcggcttcggccttcgccagcacctaccgccagcagccctcctacccgcagcagttcccctacccgcaggcgcccttcgtcagccagtacgagaactacgaccccgcgtcgcggacctacgaccagggtttcgtgtactaccggcccgcgggcggcggcgtgggcgcgggggcggcggccgtggcctcggcgggggtcatctacccctaccagccccgggcgcgctacgaggagtacggcggcggcgaagagctgcccgagtacccgcctcagggcttctacccggcccccgagaggccctacgtgccgccgccgccgccgccccccgacggcctggaccgccgctactcgcacagtctgtacagcgagggcacccccggcttcgagcaggcctaccctgaccccggtcccgaggcggcgcaggcccatggcggagacccacgcctgggctggtacccgccctacgccaacccgccgcccgaggcgtacgggccgccgcgcgcgctggagccgccctacctgccggtgcgcagctccgacacgcccccgccgggtggggagcggaacggcgcgcagcagggccgcctcagcgtaggcagcgtgtaccggcccaaccagaacggccgcggtctccctgacttggtcccagaccccaactatgtgcaagcatccacttatgtgcagagagcccacctgtactccctgcgctgtgctgcggaggagaagtgtctggccagcacagcctatgcccctgaggccaccgactacgatgtgcgggtgctactgcgcttcccccagcgcgtgaagaaccagggcacagcagacttcctccccaaccggccacggcacacctgggagtggcacagctgccaccagcattaccacagcatggacgagttcagccactacgacctactggatgcagccacaggcaagaaggtggccgagggccacaaggccagtttctgcctggaggacagcacctgtgacttcggcaacctcaagcgctatgcatgcacctctcatacccagggcctgagcccaggctgctatgacacctacaatgcggacatcgactgccagtggatcgacataaccgacgtgcagcctgggaactacatcctcaaggtgcacgtgaacccaaagtatattgttttggagtctgacttcaccaacaacgtggtgagatgcaacattcactacacaggtcgctacgtttctgcaacaaactgcaaaattgtccaatcctga

SEQ ID NO: 8 - DNA, 20 bp, artificial sequence
  amorce
gacataaccgacgtgcagcc

SEQ ID NO: 9 - DNA, 20 bp, artificial sequence
  amorce
atccacgttcgctccctgag

SEQ ID NO: 10 - DNA, 20 bp, artificial sequence
  amorce
gtatatacccaggtggcgtg

SEQ ID NO: 11 - DNA, 21 bp, Artificial Sequence
  amorce
cgaactttgctgctgctttag

SEQ ID NO: 12 - DNA, 23 bp, artificial sequence
  amorce
ggtggccgacccctactacatcc

SEQ ID NO: 13 - DNA, 26 bp, artificial sequence
  amorce
gcaaatcgcctctggtagccatagtc

SEQ ID NO: 14 - DNA, 18 bp, artificial sequence
  amorce
gtggagagtactggattg

SEQ ID NO: 15 - DNA, 18 bp, artificial sequence
  amorce
tcgtgcagccatcgacag

SEQ ID NO: 16 - DNA, 26 bp, artificial sequence
  amorce
ttggatccagcgtaggcagcgtgtac

SEQ ID NO: 17 - DNA, 25 bp, artificial sequence
  amorce
aaactcgagcatcgtagtcggtggc

SEQ ID NO: 18 - DNA, 29 bp, artificial sequence
  amorce
tcggatccggctactcgacatctagagcc

SEQ ID NO: 19 - DNA, 27 bp, artificial sequence
  amorce
gtcctcgagaccgtactggaagtagcc

SEQ ID NO: 20 - DNA, 29 bp, artificial sequence
  amorce
ttggatccgtgcagaagatgtccatgtac

SEQ ID NO: 21 - DNA, 29 bp, artificial sequence
  amorce
tttctcgaggctgggtaagaaatctgatg

SEQ ID NO: 22 - DNA, 28 bp, artificial sequence
  amorce
cactatggatcccttgatgccaacaccc

SEQ ID NO: 23 - DNA, 27 bp, artificial sequence
  amorce
cacgacctttaggatatcgtttccagg

SEQ ID NO: 24 - DNA, 21 bp, unknown
  amorce PCR OX64
acgtacgtgcagaagatgtcc

SEQ ID NO: 25 - DNA, 21 bp, unknown
  amorce PCR OX65
ggctgggtaagaaatctgatg

SEQ ID NO: 26 - DNA, 20 bp, unknown
  amorce PCR ELA1
gtatatacccaggtggcgtg

SEQ ID NO: 27 - DNA, 21 bp, unknown
  amorce PCR ELA2
cgaactttgctgctgctttag

SEQ ID NO: 28 - DNA, 19 bp, unknown
  amorce PCR 30L1
gacttcggcaacctcaagc

SEQ ID NO: 29 - DNA, 20 bp, unknown
  amorce PCR 30L2
tgttgcagaaacgtagcgac

SEQ ID NO: 30 - DNA, 20 bp, unknown
  amorce PCR actine U
gtggggcgccccaggcacca

SEQ ID NO: 31 - DNA, 24 bp, unknown
  amorce PCR actine D
ctccttaatgtcacgcacgatttc